SEQUENCE LISTING (10 sequences)

SEQ ID NO: 1 - DNA, 35 bp, Artificial sequence
  Oligonucleotide primer
gccgcggcggaggaaggcggtgaagaacgggcagg

SEQ ID NO: 2 - DNA, 35 bp, Artificial sequence
  Oligonucleotide primer
cctgcccgttcttcaccgccttcctccgccgcggc

SEQ ID NO: 3 - DNA, 25 bp, Artificial sequence
  Oligonucleotide primer
agatctgggatctccatcttcaatg

SEQ ID NO: 4 - DNA, 25 bp, Artificial sequence
  Oligonucleotide primer
cagatctccgatgagcttgaagaag

SEQ ID NO: 5 - DNA, 19 bp, Artificial sequence
  Oligonucleotide primer
ggctcccaacaccgctaac

SEQ ID NO: 6 - DNA, 21 bp, Artificial sequence
  Oligonucleotide primer
gatgttctgggagccctcagt

SEQ ID NO: 7 - DNA, 19 bp, Artificial sequence
  Oligonucleotide primer
cagatgactttcctccgtc

SEQ ID NO: 8 - DNA, 19 bp, Artificial sequence
  Oligonucleotide primer
catcacccgctcgcaatac

SEQ ID NO: 9 - DNA, 20 bp, Artificial sequence
  Oligonucleotide primer
ccggctgcgtgactgtagta

SEQ ID NO: 10 - DNA, 18 bp, Artificial sequence
  Oligonucleotide primer
accatcagaactccggct

(19) United States
(12) Patent Application Publication
(10) Pub. No.: US 2005/0009868 A1
(43) Pub. Date: Jan. 13, 2005

(54) Retinoid receptor pan-antagonists for stimulating chondrogenesis

(76) Inventors: Michael Tully Underhill, Ontario (CA); Andrea D Weston, Ontario (CA)

Correspondence Address: MYERS BIGEL SIBLEY & SAJOVEC, PO BOX 37428, RALEIGH, NC, 27627, US

(21) Appl. No.: 10/489,750

(22) PCT Filed: Sep. 17, 2002
(86) PCT No.: PCT/CA02/01421

Related U.S. Application Data
(60) Provisional application No. 60/322,874, filed on Sep. 17, 2001.

(51) Int. Cl. A61K 38/18; A61K 31/66; A61K 31/56; A61K 31/382; A61K 31/353

(52) U.S. Cl. 514/312; 514/431; 514/456; 514/12; 514/171; 514/102; 514/690; 514/167; 424/450

(57) ABSTRACT

The invention relates to methods and compositions for inducing or enhancing chondrogenesis in vivo and/or ex vivo. More specifically, the invention is directed to the use of RAR pan-antagonist compositions for the treatment, repair and engineering of cartilage.

### FIELD OF THE INVENTION

The invention relates to methods and compositions for inducing or enhancing chondrogenesis in vivo and/or ex vivo. More specifically, the invention relates to the use of RAR pan-antagonist compostions for the treatment, repair and engineering of cartilage.

### BACKGROUND OF THE INVENTION

The retinoids have been known for decades to have a considerable influence on cartilage formation. An imbalance in vitamin A metabolites, such as retinoic acid (RA), during development will result in severe skeletal defects among a multitude of other developmental anomalies (Hale, 1935; Warkany and Schraffenberger, 1946; Cohlan, 1953; Wilson et al., 1953; Kalter and Warkany, 1961). Modulation of RA availability during the time period of chondrogenesis has the most profound impact on the skeleton, suggesting that this period of skeletal development is particularly sensitive to the retinoids (Kochhar, 1973; Kwasigroch and Kochhar, 1980). Accordingly, retinoids have been shown by several groups to inhibit chondrogenesis in vivo and in vitro (Underhill and Weston, 1998).

Of the intracellular retinoid binding proteins, nuclear receptors are thought to mediate most of RA's effects on cell behaviour. Two. subfamilies of nuclear retinoid receptors exist: the retinoic acid receptors (RARs) and the retinoid X receptors (RXRs). Within each subfamily there are three subtypes (α, β, and γ), with multiple isoforms of each. These receptors belong to the steroid hormone family of nuclear receptors, and provide a level at which much of the diversity of retinoid responses is generated (Leid et al., 1992). Ligand binding to the RARs followed by recruitment of transcriptional co-activators is the basic mechanism underlying RAR-mediated gene transcription.

The RARs and their isoforms exhibit dynamic expression patterns throughout development (Mollard et al., 2000). With respect to skeletal development in the limb, RARα is expressed throughout the limb mesenchyme early in limb development. As cells begin to differentiate into chondrocytes, RARα is downregulated, remaining highly expressed in the perichondrium and in the interdigital region, whereas RARγ expression becomes localized to the cartilaginous elements (Dolle et al., 1989; Mendelsohn et al., 1991; Cash et al., 1997; Mollard et al., 2000). Throughout limb morphogenesis, RARα is expressed in noncartilage-forming regions such as the interdigital region (Mendelsohn et al., 1991). The continued expression of RARβ in prechondrogenic cells has been demonstrated to prevent their differentiation, resulting in severely malformed skeletal elements in transgenic mice (Cash et al., 1997; Weston et al., 2000).

Previous studies on the effects of RAR antagonists on chondrogenesis has been very contradictory, as some suggested that RA was needed for formation of cartilage, whereas others showed that activation of RAR-mediated signalling inhibited chondrogenesis. The Applicant's have previously demonstrated that the continued expression of RARα activity in transgenic mice was found to inhibit the chondroprogenitor-to-chondroblast transition. Likewise, inhibition of RAR using the antagonist, AGN194301, induced differentiation in primary limb mesenchymal cultures earlier than normal, resulting in a substantial increase in the number of cartilage nodules that form in these cultures confirmed by the AGN194301-induced increase in expression of cartilage-specific genes such as Col2a1 (Weston et al., 2000). Based on these findings the Applicant proposed the use of RAR subtype selected antagonist compositions for the stimulation of chondrogenesis (PCT CA99/01106 filed Nov. 19, 1999).

Upon further investigation of the mechanism underlying retinoid receptor regulation of the prechondroblast-to-chondroblast transition the Applicant focused on the role of Sox9 to identify factors downstream of RAR activity that may mediate the effects of RAR activation or inhibition. Sox9 is a transcription factor known to play an essential role in establishing the precartilaginous condensations and in initiating chondroblast differentiation (Bi et al., 1999; Smits et al., 2001). Sox9 binds to a region within the first intron of the type II collagen gene (Col2a1) to regulate its transcription (Lefebvre et al., 1996). Mutations in Sox9 underlie the rare congenital dwarfism syndrome, campomelic dysplasia (Foster et al., 1994; Wagner et al., 1994). Sox9-null mice are embryonic lethal, while Sox9-l-cells in chimeric embryos are excluded from all cartilages (Bi et al., 1999). Conversely, if ectopically expressed, Sox9 will induce ectopic Col2a1 expression and cartilage formation (Bell et al., 1997; Healy et al., 1999). To reproduce the pattern of Col2a1 expression in the cartilage elements with a reporter gene, only a small (48 bp) enhancer element from the first intron of Col2a1 containing Sox9 binding sites is required.

The Applicant has now demonstrated that Sox9 expression is regulated by the retinoid signaling pathway. In other words, RAR-mediated repression is required for induction of Sox9 (Weston et al., The Journal of Cell Biology, Volume 158, Jul. 8, 2002, pages 39-51). Moreover, expression of a dominant-negative RAR leads to an increase in Sox9 reporter activity that is substantially greater than that elicited by any other known factors. Furthermore, the p38 MAPK and PKA signaling cascades have been shown to be required downstream of retinoid signaling for chondroblast differentiation.

It is during this characterization of the hierarchical network whereby RAR-mediated signalling functions upstream of the p38 MAPK and PKA signalling pathway leading to increased Sox9 activity and regulate emergence of the chondroblast phenotype that it was surprisingly demonstrated that pan-antagonism of RARαβ_{χ} leads to an unexpectedly high level of chondrogenesis leading the way to the development of more effective compositions and methods to stimulate chondrogenesis.

### SUMMARY OF THE INVENTION

The present invention is directed to compositions and methods for stimulating chondrogenesis in vitro and in vivo. The Applicant has surprisingly demonstrated that pan-antagonism of each of the three subtypes of the retinoid receptor RARα, RARβ and RARγ (for example, by a compound able, or combination of compounds collectively able, to lessen or block the ligand-mediated biological activity, both transactivational and non-genomic) results in the effective stimulation of chondrogenesis. As shown herein, a loss in RARαβγ activity induces Sox9 expression. Sox9, which is a major regulator of cartilage formation, in turn stimulates the transactivation of the cartilage-specific gene col II leading to the stimulation of chondrogenesis.

As used herein “chondrogenesis” is defined as the development/formation of cartilage and includes the transformation of precursor cells of chondrocyte lineage into chondroblasts.

As used herein “RAR pan-antagonists” are defined as any compound having a high binding affinity to each of RARα, RARβ and RARγ such that agonist-mediated signalling is essentially inhibited and/or RAR mediated repression is enhanced leading to increased chondrogenesis.

As used herein “RARα antagonist” is any compound having a high binding affinity to RARα such that agonist mediated signalling is essentially inhibited and/or RARα mediated repression is enhanced leading to increased chondrogenesis.

As used herein “RARβ antagonist” is any compound having a high binding affinity to RARβ such that agonist mediated signalling is essentially inhibited and/or RARβ mediated repression is enhanced leading to increased chondrogenesis.

As used herein “RARγ antagonist” is any compound having a high binding affinity to RARγ such that agonist mediated signalling is essentially inhibited and/or RARγ mediated repression is enhanced leading to increased chondrogenesis.

As used herein “RAR pan-antagonist composition” is defined as any composition that effectively binds with high affinity binding to RARα, RARβ and RARγ such that RAR-mediated signalling is essentially inhibited and/or RAR mediated repression is enhanced leading to increased chondrogenesis. Such compositions may comprise;
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; or
iii) a mixture of i) and ii).

The present invention now provides surprisingly more effective therapeutic compositions and methods for the treatment of disorders involving abnormal cartilage formation and associated abnormal skeletal development resulting from disease or due to trauma. The compositions and methods generally involve the stimulation of cartilage formation and may be used/performed in vitro, in vivo and/or ex vivo.

Aspects of the methods incorporating the RAR pan-antagonist compositions of the invention may include but are not limited to;
treating damaged cartilage and associated bone in a subject;
stimulating cartilage repair and formation.
producing cartilage at a cartilage defect site in vivo;
enhancing osseous integration of orthopedic or dental implants;
treating arthritis in a subject;
use with implantable biocompatible carriers;
producing chondrocytes from precursor cells;
use with implantable prosthetic devices for repairing cartilage-associated orthopedic defects, injuries or anomalies;
promoting in vivo integration of an implantable prosthetic device into a target cartilage tissue;
repairing large segmental skeletal gaps and non-union fractures;
ex vivo tissue engineering of cartilage;
aiding the attachment of an implantable prosthesis to a cartilage site; and
maintaining the long term stability of the prosthesis in a vertebrate.

According to an aspect of the present invention is a RAR pan-antagonist composition selected from the group consisting of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii),
wherein said composition additionally comprises a pharmaceutically acceptable carrier and wherein said composition is administered to a population of cells to essentially inhibit RAR mediated signalling and/or enhance RAR mediated repression leading to increased chondrogenesis.

According to another aspect of the present invention is the use of a RAR pan-antagonist composition selected from the group consisting of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii),
wherein said composition additionally comprises a pharmaceutically acceptable carrier and wherein said composition is administered to a population of cells to essentially inhibit RAR mediated signalling and/or enhance RAR mediated repression leading to increased chondrogenesis, said use being for a method selected from the group consisting of:
a) promoting in vivo integration of an implantable prosthetic device, into a target cartilage tissue of a vertebrate by providing said RAR pan-antagonist composition on a surface of the prosthetic device and implanting the device in a vertebrate at a site where the target cartilage tissue and surface of the prosthetic device are maintained at least partially in contact for a time sufficient to permit enhanced tissue growth between the target cartilage tissue and the device; and
b) aiding the attachment of implantable prosthesis at cartilageous sites and for maintaining the long term stability of the prostheses in vertebrates by coating selected regions of an implantable prosthesis with said RAR pan-antagonist composition and implanting the coated prosthesis into a cartilageous site wherein such implantation promotes the formation of new cartilage tissue.

According to another aspect of the present invention is a method of stimulating the formation of cartilage in a subject in need of such by administering to said subject a pharmaceutically effective amount of a RAR pan-antagonist composition.

According to another aspect of the present invention is a method of stimulating chondrogenesis comprising providing a therapeutically effective amount of a RAR pan-antagonist composition selected from the group consisting of;
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii), wherein said composition essentially suppresses RARα, RARβ and RARγ activity.

In one embodiment said composition comprises the compound AGN 194310, which is an RAR pan-antagonist.

According to yet another apsect of the present invention is a method of treating arthritis in a subject, comprising providing a therapeutically effective amount of a RAR pan-antagonist composition to said subject.

According to another aspect of the present invention is a method of stimulating the expression or activity of Sox9 comprising administering a composition comprising one of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ;
iii) a mixture of i) and ii),
   to a cell containing a nucleic acid encoding Sox9. In one embodiment of this method, the RAR pan-antagonist is AGN 194310.

In another embodiment the invention comprises a method of treating a subject having a pathological condition characterized by a lack of cartilage, comprising administering to said subject a pharmaceutically effective amount of a RAR pan-antagonist composition, wherein said composition essentially inhibits or blocks RARα, RARβ and RARγ activity.

According to still a further aspect of the present invention is an implantable prosthetic device for repairing an orthopedic defect, injury or anomaly in a vertebrate, said device comprising:
(a) a prosthetic implant having a surface region implantable adjacent or within a target tissue; and
(b) RAR pan-antagonist composition incorporated on and/or within said prosthetic implant, said composition selected from the group consisting of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii),
wherein said composition additionally comprises a pharmaceutically acceptable carrier and wherein said composition essentially inhibits RAR mediated signalling and/or enhances RAR mediated repression leading to increased chondrogenesis.

According to yet another aspect of the present invention is a method for the ex vivo engineering of chondrocytes, said method comprising:
(a) culturing a population of precursor cells of chondrocyte lineage with a composition selected from the group consisting of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii),
for a time sufficient to stimulate chondrogenesis; and
(b) implanting the cells from (a) directly into a desired site in a subject or applying the cells from (a) to a device selected from the group consisting of implantable mechanical physical device, implantable biodegradable carrier; implantable biodegradable synthetic carrier, implantable prostheses, implantable demineralized allogenic bone and implantable demineralized xenogenic bone, prior to implantation into a subject.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### BRIEF DESCRIPTION OF THE DRAWINGS

The present invention will become more fully understood from the description given herein, and from the accompanying drawings, which are given by way of illustration only and do not limit the intended scope of the invention.

FIG. 1 shows that the inhibition of RAR activity enhances Sox9 activity and expression. Activation of the retinoid receptors in primary limb mesenchymal cultures with either at-RA, or the RARα specific agonist AGN193836 (836) attenuated the activity of the pGL3(4×48) Sox9 reporter (A), however, at-RA appears to be a more effective inhibitor. In contrast, inhibition of RAR activity by the RARα-specific antagonist, AGN194301 (301), or with the RAR pan-antagonist AGN194310 (310) enhances reporter activity with 310 being more potent than 301. The effects of these compounds on activity of pGL3 (4×48) are inversely proportional to their ability to activate a luciferase reporter containing an RA response element (pW1-βRARE₃-Luc) (B). The concentration-dependent effects of each compound on Sox9 reporter activity also corresponds with the ability of each compound to enhance or inhibit cartilage formation in vitro, as indicated by alcian blue staining (C). In response to AGN194301, there is an increase in Sox9 mRNA as early as day 2, but this increase appears to be transient (D). Bar: 1.5 mm. [ANOVA's (A and B) P<0.0001; Bonferroni post-tests indicate significant differences in Sox9 reporter activity at concentrations ≧5×10⁻⁹ M for 310; 1×10⁻⁷ M for 301; 5×10⁻⁸ M for 836; and 5×10⁻⁹ M for at-RA. Significant changes in activity of the RARE reporter are induced by concentrations ≧5×10⁻⁹ for 310; 1×10⁻⁷ M for 301, 5×10⁻⁹ M for at-RA; and 5×10⁻⁹ M for 836, will all P values at least <0.05].

FIG. 2 shows that Sox9 transactivation of Col2a1 is inversely associated with RAR activity. Constructs containing constitutively active receptors (RARαVP16 and RXRαVP16), or dominant-negative versions of the receptors (dnRARα and dnRXRα) were used and substantially altered the activity of the pW1-βRARE₃-Luc reporter (A, B). RARαVP16 and RXRαVP16, which enhance activity of the RARE reporter (A), attenuated activity of the pGL3(4×48) reporter (C). In contrast, the dnRARα and dnRXRα, which suppress RARE reporter activity (B) substantially activated pGL3(4×48), albeit the dnRARα has a more dramatic effect (D). [ANOVAs (A-D), P<0.0001; Bonferroni post-tests (A-D):D):*P<0.001)].

FIG. 3 shows that Sox9 binding sites are essential for dnRARα-induced reporter activity. Reporters with varying sensitivities to Sox9 were used to follow their response to the dnRARα. All reporter constructs were co-transfected with pcDNA3-hSox9 (A) or with dnRARα (B). Of four reporters analyzed, 4×48-p89 and pGL3(4×48), were most sensitive to Sox9 (A) and also exhibited the greatest response to dnRARα (B). In contrast, pGL3(−89+6) containing only the minimal Col2a1 promoter with no Sox9 binding sites, exhibited no activity in response to Sox9 (A) and was unaffected by the dnRARα (B). A reporter (pW1-Col2-Luc) containing two tandem repeats of a larger intron-1 segment of Col2a1 (including Sox9 binding sites) along with a promoter fragment, was only mildly sensitive to Sox9 (A) and was activated to a much weaker extent by dnRARα (B) compared to the 4×48-containing reporters. All reporter inductions by hSox9 or dnRARα were normalized to basal levels of respective reporters. [ANOVAs (A an B), P<0.0001, Bonferroni post-tests: *P<0.001].

FIG. 4 shows the induction of Sox9 reporter activity by dnRARα in different cells compared to vector-transfected (−) controls. The effect of dnRARα on Sox9 reporter activity was consistent in chondrogenic cells, as considerable increases in pGL3(4×48) were induced not only in limb mesenchymal cells, but also in rat articular chondrocytes and in C5.18 cells, which both have chondrogenic capacity. In contrast, no noticeable change in reporter activity is induced in the non-chondrogenic COS P7 cells. (Student's t-tests: *P<0.001).

FIG. 5 shows histone deacetylase-mediated gene repression is required for chondrogenesis. The effects of TSA on Sox9 reporter activity in the presence or absence of AGN194310 (A) and on pW1-βRARE₃tkLuc (B) were analyzed. TSA attenuated Sox9 reporter activity in a concentration-dependent manner, and inhibited the effects of AGN194301 (A). In contrast, TSA enhanced activity of the pW1-αRARE₃tkLuc reporter in a concentration-dependent manner (B). The inhibition in Sox9 reporter activity correlates with the decrease in the number of cartilage nodules forming in response to TSA, as seen in day 4 alcian blue-stained cultures (C). The increases in Sox9 reporter activity induced by co-transfection with dnRARα, or by treatment with AGN194301 or AGN194310 are attenuated by co-expression of pCMX-GAL4/N-CoR (D). Bar: 1.5 mm. [ANOVAs (A and B) P<0.0001 for all cases; Bonferroni post-tests (A and B): * P<0.01, **P<0.001, ***P<0.0001 all versus respective control cultures; Student's t-tests (D); **P<0.001, ***P<0.0001].

FIG. 6 shows the p38 MAPK pathway and the PKA pathway are activated in response to RAR inhibition. Reporters containing a cAMP response element (pCRE-TA-Luc) or activator protein-1 response element (pAP-1-TA-Luc) are both activated in response to co-transfection with dnRARα (A and B). Co-transfection with dnRARα also induces transactivation of a GAL4 reporter (pG5-Luc) by the transcription factors ATF2 and CREB, both of which are fused to the DNA binding domain of GAL4 (FA-ATF2 and FA-CREB) (C and D). The ability of PKAc and MKK6E to activate FA-CREB and FA-ATF2, respectively, was tested for positive control purposes. [Student's t-tests (A and B); *P<0.0005; ANOVAs (C and D) P<0.0001; Bonferroni post-tests (C and D), #P<0.0001, **P<0.001, ***P<0.01].

FIG. 7 shows the inhibition of p38 and PKA prevents chondrogenesis. In the presence of 5 or 10 μM SB202190, there was a decrease in Sox9 reporter activity compared to untreated controls (A). SB202190 also attenuated the chondrogenic response to AGN194301 and the dnRARα (A). This inhibition was reflected by a lack of cartilage formation in vitro, as almost no cartilage nodules form in response to 10 μM SB202190 (D) in contrast to the presence of numerous nodules in untreated control cultures (C). Similar to SB202190, the PKA inhibitor H89 reduced Sox9 reporter activity both in the presence or absence of AGN194301 or dnRARα (B). In H89-treated cultures (10 μM) (F), fewer nodules are detected compared to untreated cultures (E); these nodules are much smaller and stain only weakly with alcian blue. Bar: 1.5 mm. [ANOVA's (A and B) P<0.0001; Bonferroni post-tests (A and B): *P<0.05, **P<0.01, ***P<0.001, #P<0.005, all versus respective non-SB202190 or non-H89-treated controls].

FIG. 8 shows effects of different components of the p38 signaling cascade on ATF2 induction. Transient transfection with MKK6E induced ATF2 activity greater than 2-fold, however, transient expression of p38α or p38β either alone, or in combination, had no appreciable effect on FA-ATF2 activity (A). When co-transfected with MKK6E, however, p38α or p38β enhance FA-ATF2 activity considerably, and when both isoforms are transfected together along with MKK6E, there was an even greater induction of FA-ATF2 (A). The effects of MKK6E, p38α, and p38β on Sox9 reporter activity corresponds with their ability to activate FA-ATF2 (B). MKK6E induced an almost 2.5-fold increase in activity of pGL3(4×48), while p38α and p38β alone had no noticeable effect. When co-transfected with MKK6E, p38α and p38β each enhanced Sox9 reporter only slightly, but when transfected together along with MKK6E, the increase in Sox9 transactivation was greater than 4-fold (B). PKAc induces FA-CREB activity by greater than 30-fold, (C) but only enhanced Sox9 reporter activity slightly (by less than 2-fold) (D). [ANOVAs (A and B) P<0.0001; Bonferroni post-tests (A): *P<0.001, (B) *P<0.001; Student's t-test (C): **P<0.0005, (D) *P<0.001].

FIG. 9 shows Sox9 expression and activity measured in response to manipulation of the PKA signaling pathway. In the absence of exogenous Sox9, addition of pCPT-cAMP (500 μM) or co-transfection with dnRARα increases Sox9 reporter activity, while H89 (10 μM) repressed reporter activity in primary limb mesenchymal cells (A). In the presence of co-transfected Sox9, reporter activity was elevated >100 fold, and the addition of pCPT-cAMP or co-transfection with dnRARα only has a small stimulatory effect (<1.5 fold), while the addition of H89 slightly decreased reporter activity. The response of the mutant Sox9-181A was similar to wild-type Sox9 (A). To demonstrate that the mutation functioned as expected, COS P7 cells were transfected with wild-type Sox9 or the mutant Sox9 in the presence or absence of PKA (B). The presence of PKA led to a >1.5 fold induction in reporter activity, whereas the mutant Sox9 exhibited little increase with co-transfected PKA. Modulation of PKA signaling influenced Sox9 and Col2a1 expression in primary limb mesenchymal cells (C). Real-time quantitative PCR was used to demonstrate that activation of PKA led to an increase in Sox9 and Col2a1 mRNA abundance, whereas treatment with H89 suppressed their expression. [ANOVA's (A-C) P<0.0001; Bonferroni post-tests (A-C): *P<0.05, **P<0.01, ***P<0.001, all versus respective non-treated controls].

FIG. 10 shows the ability of ATF2 and CREB to reverse the effects of RARαVP16 on Sox9 reporter activity. MKK6E partially prevented the inhibitory response of RARαVP16 whereas p38α and p38β alone, or in combination had little if any effect (A). Co-transfection with MKK6E and p38α or p38β was able to partially restore Sox9 activity, whereas co-transfection of MKK6E with both p38α and p38β completely restored the effects of RARαVP16 (A). Activation of PKA by transient transfection with PKAc was also able to restore Sox9 reporter activity to basal levels in RARαVP16-transfected cultures. [ANOVA (A and B) P<0.0001; Bonferonni post-tests: (A) *P<0.001; (B) *P<0.001].

### DETAILED DESCRIPTION OF THE PREFERRED EMBODIMENTS

The Applicants have identified a pathway of molecular regulation of chondroblast differentiation leading to chondrogenesis. More specifically the Applicant has demonstrated that essentially blocking and/or enhancing RAR mediated repression of RARα, RARβ, and RARγ receptors leads to significantly higher level of chondrogenesis than previously demonstrated. With this knowledge, more effective RAR pan-antagonist compositions can be made and used in vitro and in vivo for the stimulation of chondrogenesis for the treatment of a variety of clinical disorders and for tissue engineering.

RAR pan-antagonists have been suggested for use for the treatment of cartilage or bone pathology characterized by ectopic endochondrial ossification (U.S. Pat. No. 6,313,168). This refers to the halting of maturation of chondrocytes to hypertrophic chondrocytes, thereby slowing or inhibiting cartilage calcification and subsequent replacement by bone. U.S. Pat. No. 6,313,168 discloses the use of antagonist compounds specifically for those clinical indications involving the hypertrophic chondrocyte response involved in bone pathologies (i.e. cartilage and bone diseases resulting from abnormal endochondral ossification). This patent does not teach or suggest any use of RAR antagonists or RAR pan-antagonists to stimulate chondrogenesis which includes the establishment of chondrocytes. Furthermore, this patent does not teach or suggest the use of RAR antagonists or RAR pan-antagonists to promote normal bone repair (ie. for spinal fusion, repair of non-union breaks, etc.) or the effective stimulation of chondrogenesis and certainly not at the high level of chondrogenesis presently demonstrated. As such the present invention is directed to the use of RAR pan-antagonist compositions for a variety of in vitro, in vivo and ex vivo applications and clinical indications not at all contemplated by U.S. Pat. No. 6,313,168.

The Applicant now demonstrates that repression of RAR-mediated signaling results in the activation of p38 MAPK and PKA signaling pathways and that the effects of RAR antagonism on chondrogenesis can be blocked by inhibition of p38 MAPK and PKA activity. Specifically, inhibition of MAPK p38α and/or MAPK p38β prevents cartilage formation and attenuates Sox9 reporter activity. Conversely, activation of the p38α or p38β pathways is sufficient to promote Sox9 activity. Moreover, activation of p38α/β or PKA signaling are the only means identified to date, with the exception of expression of Sox9, that rescue the negative effects of activation of the RAR-signaling pathway on chondrogenesis. Together, these results suggest that activation of the p38 MAPK and PKA pathways upon RAR-mediated gene repression is necessary for acquisition of the chondroblast phenotype.

Using soluble compounds in combination with expression plasmids, the Applicant has demonstrated a surprisingly tight correlation between retinoid receptor activity and Sox9 transactivation of Col2a1. The extent of RAR inhibition was found to be inversely proportional to the level of Sox9 induction. Moreover, manipulations affecting all three RARs were more influential than those affecting only the RARα subtype, indicating the importance of at least two RAR subtypes in chondrogenesis. The induction of Sox9 activity was dramatically increased upon co-transfection with dnRARα which is a dominant negative RAR. To date, no single factor has been shown to induce Sox9 to a similar extent, including numerous factors known to potently stimulate chondrogenesis such as BMP-2 and BMP-4 either in soluble form or after co-transfection of expression plasmids containing activated BMP receptors (data not shown). The ability of the dnRARα to stimulate Sox9 activity to a much greater extent than AGN194301 (RARα subtype selective antagonist) or AGN194310 (RAR pan-antagonist), is due to the constitutive repressive activity of the truncated receptor. Consistent with this, the dnRARα has previously been shown to exhibit enhanced association with nuclear co-repressors (Wong and Privalsky, 1998). The ability of the dnRXR to significantly enhance Sox9 activity may suggest the absence of the RXR AF-2 domain may facilitate formation of a RAR/dnRXR-nuclear co-repressor complex.

The more pronounced effects of AGN 194310 and at-RA compared to the more specific compounds, AGN 194301 and AGN 193836, respectively, implicates the involvement of at least two receptor subtypes in regulating chondroblast differentiation. These results are further supported by the effects of the dnRARα which broadly inhibits RAR-mediated signaling (Damm et al., 1993). Taken together, it is now demonstrated that an effective loss of the ligand-induced activity of multiple RARs initiates chondroblast differentiation at significantly high levels. Accordingly, these findings explain the failure of cells expressing a weak constitutively active RAR in transgenic mice to differentiate into chondroblasts and contribute to cartilage nodules. Continued RAR activity would result in a reduction of Sox9 expression and/or activity thereby preventing chondroblast differentiation, similar to that observed in Sox9 null cells.

Inhibition of RAR Activity Enhances Chondrogenesis Through a Sox9-Dependent Mechanism

Previously, the continued expression of RARα activity in transgenic mice was found to inhibit the chondroprogenitor-to-chondroblast transition. Likewise, inhibition of RARα using the subtype-specific antagonist, AGN194301, induced differentiation in primary limb mesenchymal cultures earlier than normal, resulting in a substantial increase in the number of cartilage nodules that form in these cultures. This induction of cartilage formation was confirmed by the AGN194301-induced increase in expression of cartilage-specific genes such as Col2a1 (Weston et al., 2000). Given that Sox9 has previously been shown to be important in regulating the expression of Col2a1, we analyzed the effects of RARα antagonism on Sox9 expression and activity in an attempt to further understand the mechanism whereby retinoid signaling regulates chondroblast differentiation.

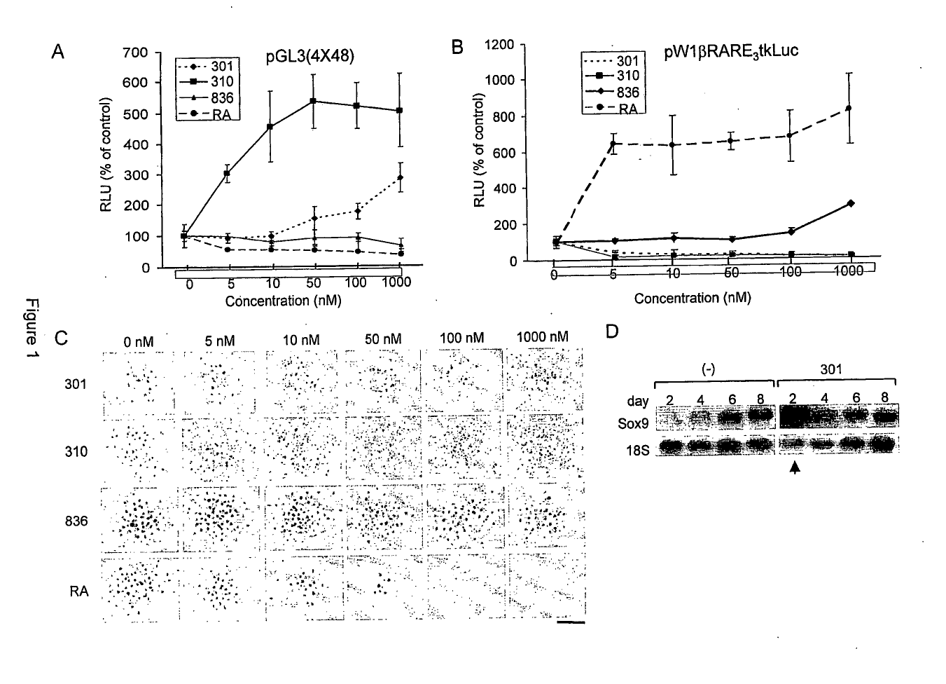

To follow endogenous Sox9 activity in primary mesenchymal cells, a reporter-based approach was used in which cells were transiently transfected with pGL3(4×48), a reporter containing four repeats of a Sox9 binding site from the first intron of Col2a1. The RARα-specific antagonist, AGN194301 (301), induced a concentration-dependent increase in reporter activity, whereas at-RA and the RARα-specific agonist, AGN193836 (836), attenuated reporter activity (FIG. 1A). Cells were treated with the RAR pan-antagonist, AGN194310 (310), concentrations as low as 10 nM induced Sox9 reporter activity greater than the maximal response elicited by higher doses of 301. The maximal response to the pan antagonist was about a 530% induction at 50 nM, whereas the greatest induction of Sox9 reporter activity by the RARα-specific antagonist was about 280% at 1 μM, a concentration at which this antagonist affects ligand binding to other RAR subtypes (Weston et al., 2000). Similar to RAR antagonism, the reduction in reporter activity caused by a pan-agonist such as at-RA was more pronounced than that induced by the RARα-specific agonist, 836. At-RA reduced reporter activity to 53% at 5 nM, while in response to a much higher dose of 836 (1 μM), reporter activity was reduced only to 64% of control. Taken together, these results indicate that a loss in activity of all RARs is more efficient at inducing cartilage differentiation than inhibition of the RARα subtype alone.

The effects of RAR modulation on Sox9 activity are opposite to that of the effects of each compound on activity of a retinoic acid responsive reporter (pW1-βRARE₃-Luc) in primary limb mesenchymal cells (FIG. 1B). For instance, at-RA activated the RARβ reporter to a greater extent than about 836, while reporter activity was attenuated by 310 more effectively than by 301. Thus, activation of the Sox9-responsive region of Col2a1 appeared to be very closely associated with the status of RAR activity. This close association was reflected in the response of primary cultures to treatment with each compound (FIG. 1C). Treatment with either at-RA, the RARα agonist, or with the antagonists for 4 days affects the formation of cartilage nodules in a manner that is consistent with their effects on Sox9 reporter activity. More specifically, at-RA is a more potent inhibitor of cartilage nodule formation than 836 while the increase in nodule formation can be observed at a lower concentration of the pan-antagonist, (10 nM 310) compared to the RARα-specific antagonist (1 βM 301). Taken together, these results validate the utility of the Sox9 reporter assay to indirectly measure the status of chondroblast differentiation and highlight the significant role of RAR-mediated signaling in regulating expression of the chondroblast phenotype.

The enhanced Sox9 reporter activity caused by RAR inhibition is due, in part, to an increase in the expression of Sox9 mRNA, as treatment of primary cultures with 1 μM 301 resulted in an precocious increase in Sox9 expression (FIG. 1D). There was a noticeable increase in Sox9 mRNA from 2-day cultures treated with 301, but this increase over control cultures was much less pronounced by days 4 and 6. Thus, inhibition of RAR activity is induced at an early, transient upregulation of Sox9 mRNA that presumably contributes to the enhanced Sox9 reporter activity seen in response to the same compound.

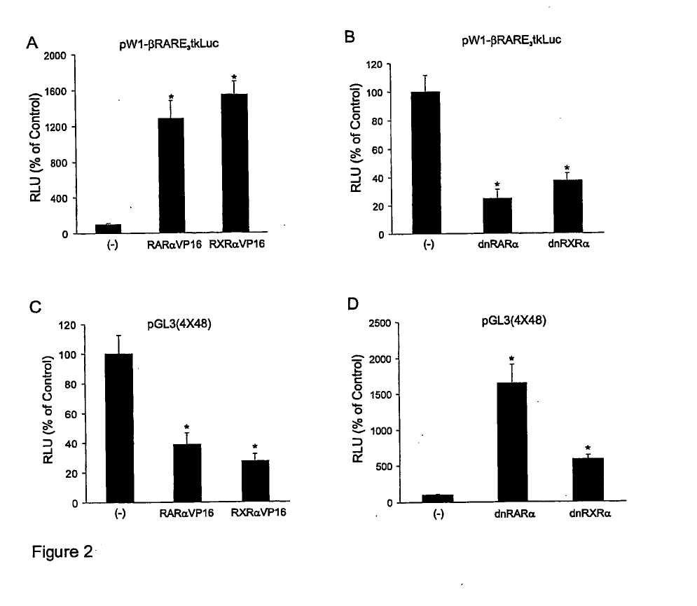

Modified versions of the RARs or RXRs were introduced into primary limb mesenchymal cultures to follow their effect on Sox9 reporter activity as this would confirm the influence of RAR activity on chondroblast differentiation. To examine the effect of RAR activation without agonist addition, constitutively active versions of RARα and RXRα were used by fusing the acidic activation domain of VP16 to the C-terminus of RAR and RXR, referred to as RARαVP16 and RXRαVP16, respectively (Underhill et al., 1994). The ability of these modified receptors to potently activate an RARE reporter in the absence of an exogenous agonist was confirmed as co-transfection of RARαVP16 and RXRαVP16 induced RARE reporter activity by 15- and 17-fold, respectively in the absence of agonist (FIG. 2A).

In addition to modifying RARE activity with constitutively active versions of the retinoid receptors, dominant-negative versions of the receptors (dnRARα and dnRXRα) were also generated and transfected into primary limb mesenchymal cultures. These dominant-negative derivatives are C-terminal truncations of RARα and RXRα that retain their ability to bind DNA and ligand, but lack the AF-2 transactivation function (Damm et al., 1993; Feng et al., 1997). When co-transfected into primary cultures, both dnRARα and dnRXRα were effective at completely blocking activity of the RARE reporter (FIG. 2B). Co-transfection of the modified receptors affected Sox9 reporter activity in a manner that is inversely proportional to their ability to trans-activate βRARE₃-tk-Luc. Both the RARαVP16 and RXRαVP16 inhibit Sox9 reporter activity, while the dnRARα and dnRXRα potently activate this reporter (FIG. 2C, D). The activation induced by co-transfection with dnRARα was more dramatic than that elicited by any other factor studied to date. Similar to the results using receptor agonists and antagonists, these results demonstrated a strong influence of retinoid receptor activity on chondrogenesis.

Chondrogenic Response to RAR Inhibition Requires 48 bp Enhancer Elements within Col2a1 and is Specific to Chondrogenic Cells

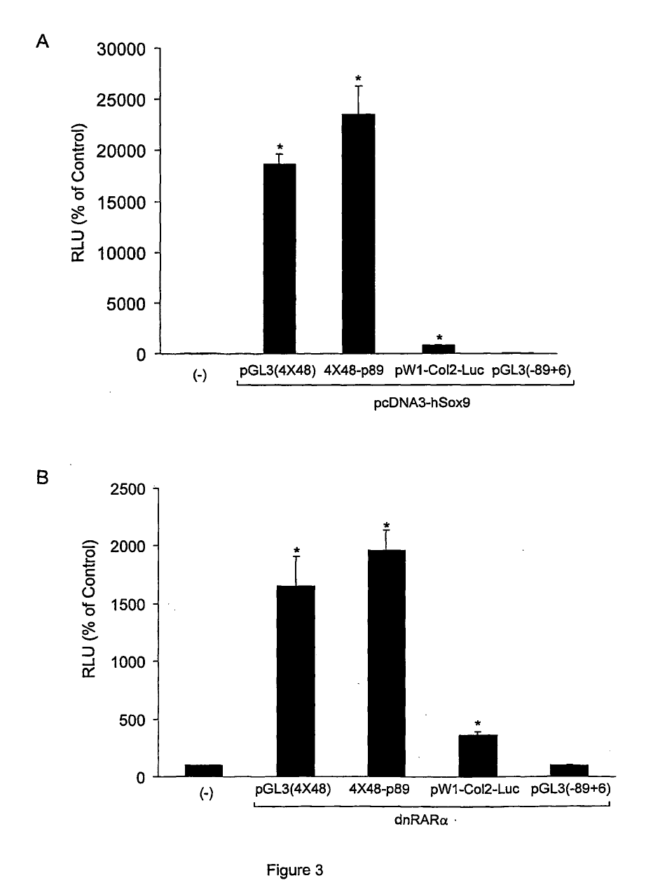

To closely examine the contribution of RAR inhibition to Sox9 activity, reporters with varying sensitivities to Sox9 were used to follow their response to the dnRARα. Of four reporters analyzed, 4×48-p89 and pGL3(4×48), which demonstrated the greatest sensitivity to Sox9 (FIG. 3A), also exhibited the greatest response to dnRARα (FIG. 3B). In contrast, pGL3(−89+6), a reporter containing only the minimal Col2a1 promoter with no 48 bp Sox9 binding sites, exhibited no activity in response to Sox9 and was unaffected by the dnRARα (FIG. 3). A reporter containing two tandem repeats of a larger intron-1 segment of Col2a1 (including Sox9 binding sites) along with a promoter fragment, was only mildly sensitive to Sox9 and was activated to a much lesser extent by dnRARα compared to the 4×48-containing reporters. These results demonstrate a direct relationship between inhibition of RAR signaling and Sox9 activity.

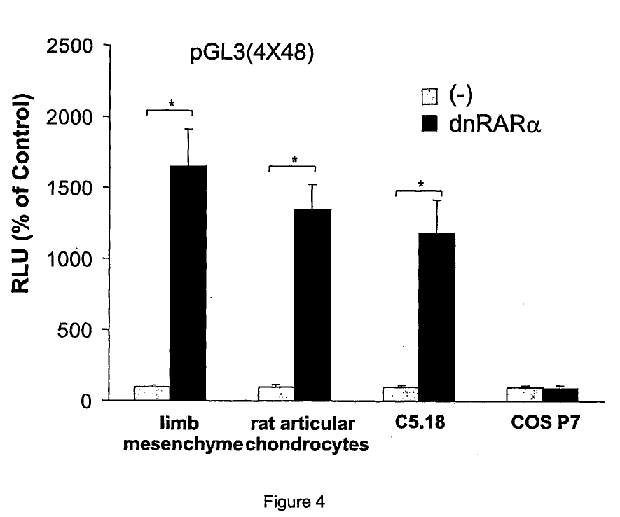

Despite the induction of Sox9 reporter activity elicited by dnRARα in other cells with chondrogenic capacity, such as de-differentiated rat articular chondrocytes, and C5.18 chondroprogenitor cells, activity of Sox9 reporter activity was not noticeably affected in COS P7 cells (FIG. 4). Given that COS P7 cells are non-chondrogenic, these results suggest that the Sox9 reporter induction caused by RAR inhibition may be restricted to cells with chondrogenic capacity.

Chondrogenesis Requires Histone Deacetylase-Mediated Gene Repression

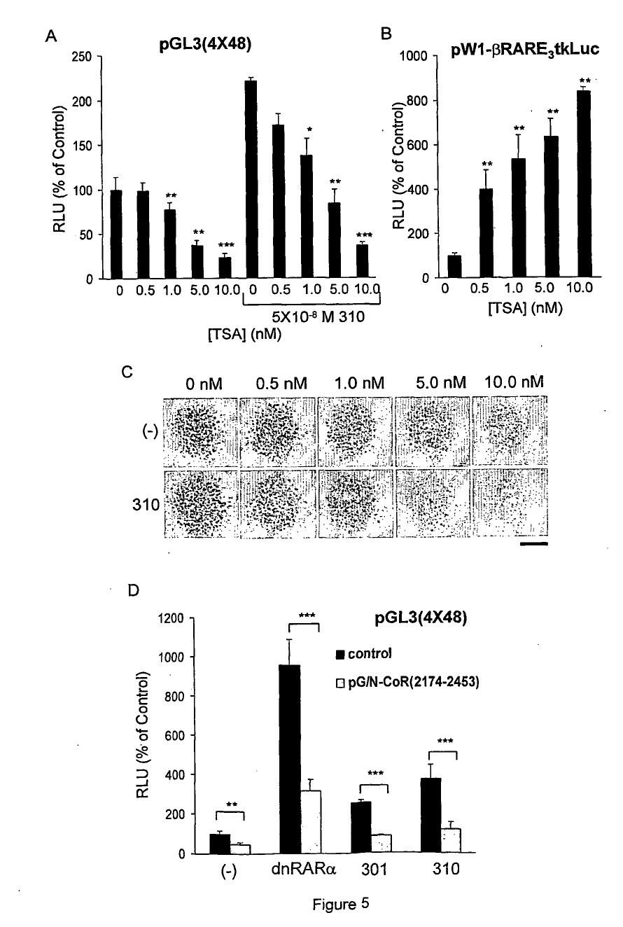

Transcriptional regulation by the retinoid receptors depends, for the most part, on ligand availability. In the absence of ligand, RAR/RXR heterodimers bind to and repress the transcription of various target genes. Receptor-mediated repression is due to association with nuclear complexes containing co-repressors (N-COR and SMRT) and histone deacetylases (HDACs) (Nagy et al., 1997). Trichostatin A (TSA) is a Streptomyces metabolite that specifically inhibits histone deacetylases leading to hyperacetylation of histones and other proteins (Finnin et al., 1999). To date, TSA has been shown to act as a potent inducer of differentiation in many cell types, some of which are also induced to differentiate by treatment with RA. Chondroprogenitors which, in contrast to most cell types, do not differentiate in response to RA also respond uniquely to TSA, as indicated by both a dose-dependent decrease in Sox9 reporter activity (FIG. 5A), and in cartilage nodule formation (FIG. 5C) in response to a TSA-induced increase in RARE reporter activity (FIG. 5B). TSA also attenuated the 310-induced increase in Sox9 reporter activity and nodule formation (FIG. 5A, C). The inhibitory effects of TSA on chondrogenesis were achieved at relatively low concentrations of TSA, as higher concentrations have been used to induce differentiation of many cell types including NIH3T3 cells and acute promyelocytic leukemia (AML) blasts (Sugita et al., 1992; Ferrara et al., 2001). Moreover, the well-characterized ability of TSA to inhibit IL-2 gene expression was found to have an IC₅₀ of 73 nM (Koyama et al., 2000), which is greater than the highest concentration (10 nM) used in the present study. These results demonstrate an important requirement for HDAC-mediated gene repression in chondroblast differentiation.

To further examine the importance of nuclear co-repressors in chondroblast differentiation the ability of a dominant-negative version of N-CoR, pCMX-G/N-CoR (2174-2453) was examined on the modulation of Sox9 reporter activity. This construct lacks the HDAC interaction domain and contains the nuclear hormone receptor interaction domain of N-CoR, a region similar to that of SMRT, which was recently shown to disrupt nuclear co-repressor function (Koide et al., 2001). Consistent with these activities, the pCMX-G/N-CoR(2174-2453) inhibited the ability of the antagonists, and the dnRAR, to decrease RARE reporter activity (data not shown). Expression of pCMX-G/N-CoR(2174-2453) alone led to an about50% decrease in basal Sox9 reporter activity. Moreover, co-expression of pCMX-G/N-CoR (2174-2453) completely inhibited the stimulatory effects of 301 and 310, and repressed the effect of the dnRAR on the Sox9 reporter (FIG. 5D). These results suggest that active repression by RARs is required for chondroblast differentiation and that this repression requires deacetylase activity.

RAR Inhibition Activates the p38 MAPK and PKA Pathways

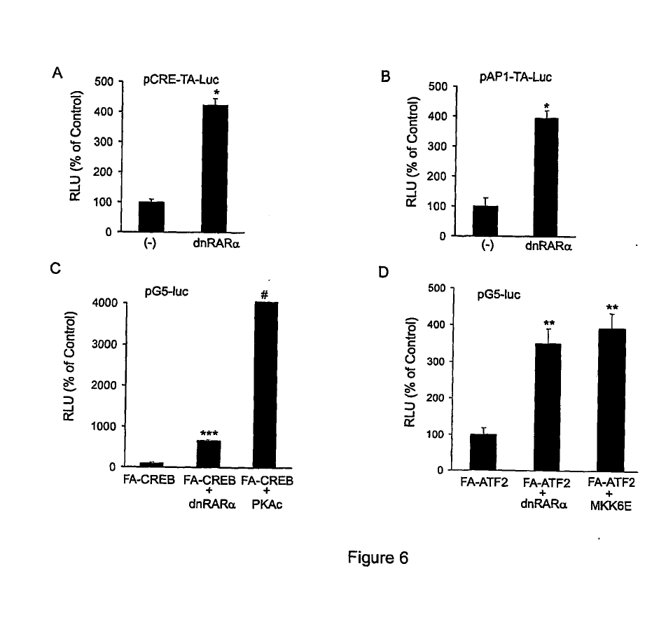

To elucidate the mechanism whereby a loss in RAR activity leads to enhanced Sox9 activity, pathway profiling vectors were used to uncover signal transduction pathways that act downstream of retinoid signaling. Various reporters containing reiterated enhancer sequences, were transiently co-transfected into primary cultures with a dnRARα. Co-transfection with the dnRARα was used as it is a potent constitutive repressor that consistently induces high Sox9 activity in primary cells. The luciferase-based reporters used contained response elements for Activating Protein-1 (pAP-1-TA-Luc), cAMP (pCRE-TA-Luc), nuclear factor of κB cells (pNFκB-TA-Luc), nuclear factor of activated T cells (pNFAT-TA-luc), serum, (pSRE-TA-Luc), glucocorticoids (pGRE-TA-Luc) and interferons (pISRE-TA-Luc). Each vector contained the reiterated response elements upstream of a TATA box and the luciferase gene. Interestingly, when co-transfected with a dnRARα, the only reporters appreciably affected (>2-fold increases) were pCRE-TA-Luc and pAP-1-TA-Luc. Co-transfection with dnRARα enhanced activity of these reporters by greater than 4-fold (FIG. 6A, B), indicating that RAR inhibition may result in activation of pathways upstream of CRE and AP-1 responses.

The protein kinase A (PKA) pathway is a predominant pathway through which genes containing a cAMP-response element are activated. When activated through various stimuli, PKA phosphorylates cAMP-response element binding protein (CREB), which binds to and activates genes containing cAMP response elements (CRE). Accordingly, co-transfection of pCMV-PKA dramatically enhanced activation of pCRE-TA-Luc (data not shown). Given that a pCRE-TA-Luc is activated in cells transfected with a dnRARα, the ability of this modified receptor was tested to induce activation of CREB. A chimeric trans-activator protein containing CREB fused to the DNA binding domain of the yeast transcriptional activator GAL4 (pFA-CREB) was transiently transfected into cells with a luciferase reporter containing a reiterated GAL4 DNA binding element. Thus, by monitoring the activity of the pG5-Luc reporter, the activation of FA-CREB was indirectly followed. Co-transfection of pCMV-PKA into the primary cultures induced an about 40-fold increase in pG5-Luc (FIG. 6C). Co-transfection with dnRARα enhanced FA-CREB-induced transactivation of pG5-Luc by approximately 6-fold.

In addition to the PKA pathway, the potential mechanisms that may underlie the activation of AP-1 by dnRARα were investigated. Activating protein-1 collectively refers to dimeric transcription factors composed of Jun, Fos, or ATF (activating transcription factor) subunits. Surprisingly, a dominant-negative version of Fos (A-Fos), which substantially diminishes pAP-1-TA-Luc reporter activity, was found to have no noticeable effect on activity of the Sox9 reporter (data not shown), suggesting that the induction of pAP-1-TA-Luc by dnRARα does not involve activation of Jun/Fos dimers. Moreover, constitutively active versions of kinases within the MAPK pathways were tested for their ability to modulate Sox9 transactivation. Of the kinases known to be upstream of AP-1 activation, only a constitutively active version of MKK6 (MKK6E) consistently led to increased Sox9 reporter activity. The predominant targets of MKK6 appear to be the p38 mitogen-activated protein kinase (MAPK) isoforms. When phosphorylated, p38 phosphorylates and activates several targets including the AP-1 component ATF2. As a positive control, MKK6E was co-transfected into cells and found to induce activity of pAP-1-TA-Luc (data not shown). Given that ATF2 has been shown to bind to AP-1 response elements, the pG5-Luc reporter was used to measure the activity of FA-ATF2, a chimeric of ATF2 and the DNA binding domain of GAL4. Co-transfection of dnRARα induced an increase in FA-ATF2-activation of pG5-Luc that was almost as robust as the induction by MKK6E (FIG. 6D).

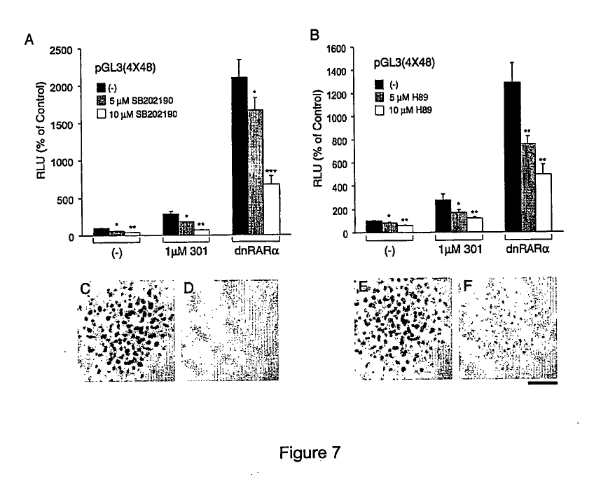

Further support for the role of p38 MAPK and PKA in chondroblast differentiation comes from the reduction in Sox9 reporter activity caused by the p38 MAPK inhibitor SB202190 and the PKA inhibitor H89 (FIG. 7A, B). These inhibitors also attenuated the induction of Sox9 reporter activity by dnRARα and by 301 (FIG. 7A, B). Consistent with this, the inhibitors at 10 μM inhibited the formation of cartilage nodules in untreated (FIG. 7 D, F) and 301-treated cultures (data not shown) compared to untreated cultures (FIG. 7C, E).

Activation of ATF2 and CREB Induces Sox9 Transactivation Response

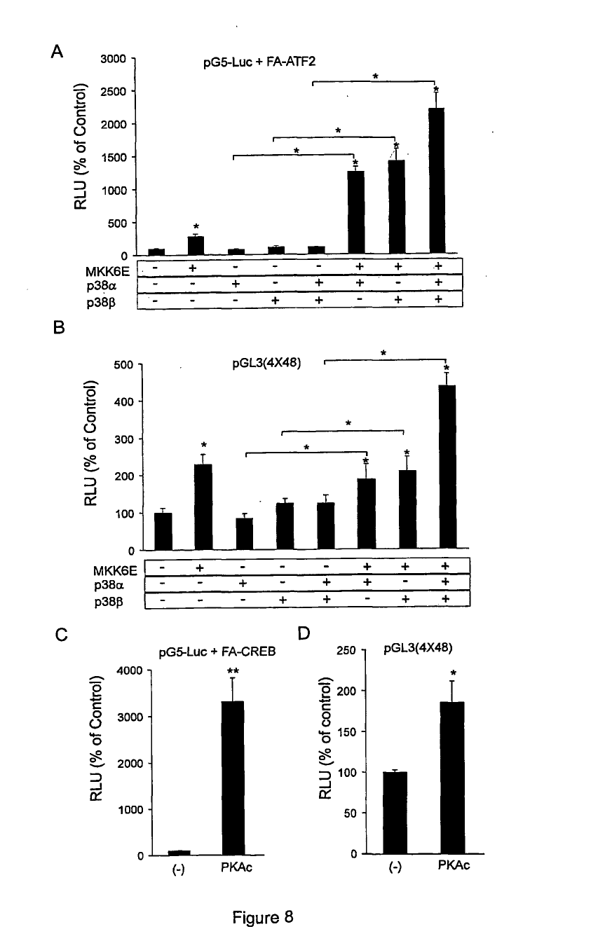

The studies described above suggest that the suppression of RAR activity leads to activation of the p38 MAPK and PKA signaling pathways. Phosphorylation of ATF2 and CREB is reflective of activation of p38 MAPK and PKA signaling pathways, respectively. To further investigate a possible role for these signaling pathways in the activation of Sox9, factors involved in these pathways were transiently transfected into the mesenchymal cells along with the Sox9 reporter. Transient transfection of a constitutively active version of MKK6 (MKK6E) induces an approximate 3-fold activation of FA-ATF2 (FIG. 8A). When transfected along with p38α or p38β, MKK6E is able to induce FA-ATF2 activity by approximately 13- and 14-fold, respectively, and even more so with the two isoforms together. P38α and p38 β alone or in combination, however, have no noticeable effect on Sox9 activity. The ability of each expression plasmid to induce activation of FA-ATF2 is directly proportional to their influence on Sox9 reporter activity (FIG. 8B), with a >4.5-fold activation by co-transfection with MKK6E along with p38α and p38β. Similarly, Sox9 is activated by the catalytic subunit of PKA, which potently enhances FA-CREB activity. The induction of Sox9 activity by PKA, however, is relatively mild given the level of FA-CREB activation elicited by PKA. These results demonstrate the relevance of activation of the p38 and PKA pathways by dnRARα, as each pathway has the potential to induce Sox9 transactivation of Col2a1.

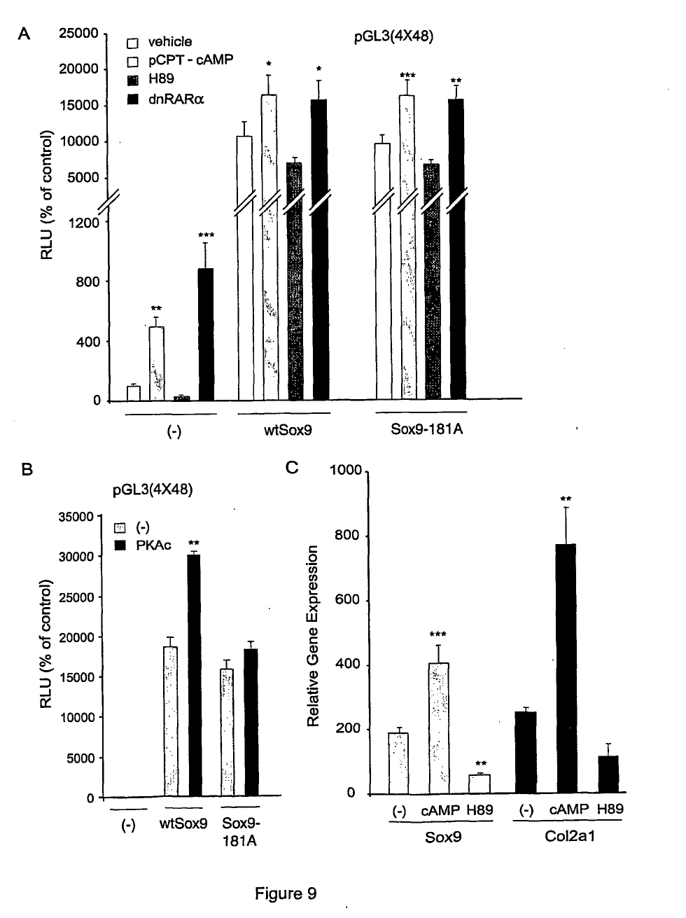

Sox9 DNA binding, and hence its transcriptional activity has been shown to be induced by PKA-mediated phosphorylation of serines 64 and 181 (Huang et al., 2000). Specifically, PKA phosphorylation of serine 181 in Sox9 was found to occur in chondrocytes of the prehypertrophic zone in response to parathyroid hormone-related peptide (Huang et al., 2000; Huang et al., 2001). To determine if the same phosphorylation event occurs in response to RAR antagonism, the ability of dnRARα to induce Sox9 reporter activity was compared in the presence of a co-transfected vector containing wtSox9 versus a mutant Sox9 in which serine 181 was replaced with alanine (Sox9-181A). In the absence of exogenous Sox9, Sox9 reporter activity was increased about 4.5 fold by activation of the PKA pathway using pCPT-cAMP (500 μM) in comparison to an about 9-fold increase by co-expression of dnRARα. Co-transfection with wt Sox9 or Sox9-181A increased reporter activity about 100 fold, and this was further increased (<1.5 fold) by the addition of pCPT-cAMP or by co-expression of PKAc or a dnRARα and decreased by the addition of H89 (FIG. 9A and data not shown). There is no significant difference in the activity of Sox9 versus Sox9-181A suggesting that phosphorylation of serine 181 was not required for Sox9 activity during chondroblast differentiation. As mentioned, immunolocalization studies detected Ser181 phosphorylated Sox9 in prehypertrophic chondrocytes. The cells used here, however are chondroprogenitors and thus, Sox9 activity may be regulated through distinct post-translational modifications within each cell type. To ensure that the mutant Sox9 functions in a manner consistent with that previously reported (Huang et al., 2000), wtSox9 and Sox9-181A were transfected into COS P7 cells in the presence or absence of an expression vector for the catalytic subunit of PKA. COS P7 cells were originally used to identify Ser181 as the PKA phosphorylation site, and as expected, the ability of PKAc to activate Sox9 in these cells is almost completely blocked by the Ser181 mutation (FIG. 9B). Thus, similar to earlier reports, Ser181 of Sox9 appears to be required for increased activation of Sox9 by PKA in these cells, but this is clearly not the case in the limb mesenchymal cells used in this study.

To determine if modulation of PKA activity affects the expression of Sox9 and Col2a1 transcripts, real-time quantitative PCR was used to measure their relative expression levels in comparison to rRNA. Sox9 and Col2a1 expression were increased by more than 2-fold in response to a 2-day treatment with pCPT-cAMP (500 μM), and decreased by more than 2-fold in response to H89 (10 μM) (FIG. 9C). A similar increase in Col2a1 expression by activation of PKA in limb mesenchymal cultures has previously been reported (Kosher et al., 1986). Our results, therefore, suggest that PKA regulates Sox9 activity during chondroblast differentiation by influencing Sox9 expression levels and not through phosphorylation of Ser181.

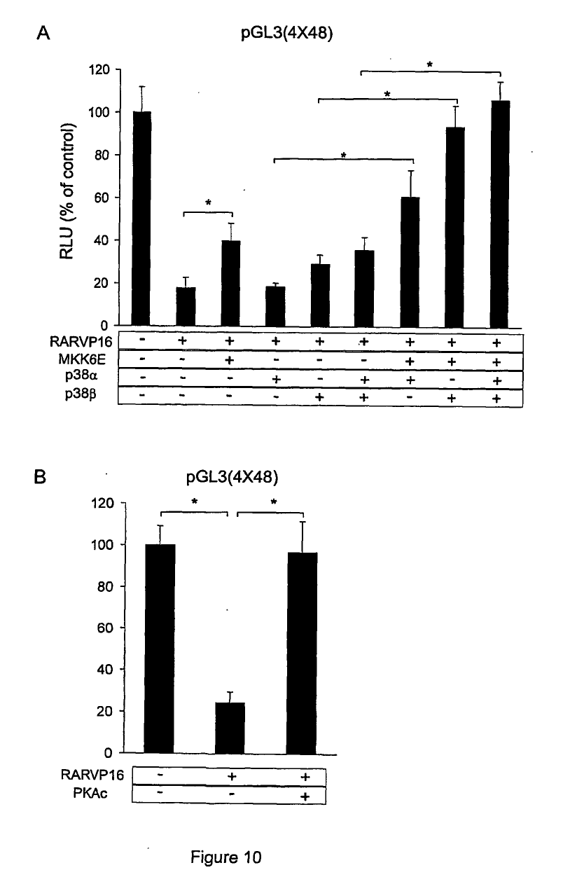

The influence of p38 MAPK and PKA signaling pathways on chondrogenesis is demonstrated by their ability to rescue the decrease in Sox9 activity induced by RARαVP16 (FIG. 10). Although Sox9 activity was only partially restored by co-transfection with RARαVP16 and MKK6E, transfection of each isoform in combination with MKK6E resulted in levels of Sox9 activity that were almost as high as those obtained in the absence of RARαVP16. Not surprisingly, MKK6E co-transfected with both isoforms of p38 (which causes the most pronounced activation of ATF2) results in a complete rescue of Sox9 activity (FIG. 10A). Similarly, PKAc can almost completely rescue the effects of RARαVP16 (FIG. 10B). These results are not due to modulation of RARαVP16, as neither MKK6E or PKAc inhibited RARαVP16 induction of an RARE reporter (data not shown).

Pan-Antagonist Compositions

With the demonstration that pan-antagonism of PAR functions to stimulate chondrogenesis, effective pan-antagonist compositions may now be used as potent stimulators of chondrogenesis and associated skeletal development. Furthermore, various therapeutic in vivo and in vitro uses of such pan-antagonist compositions are now made possible especially those uses involving the treatment of abnormal or inappropriate chondrogenesis and related skeletal development.

As previously stated, the pan-antagonist compositions of the invention effectively bind with high affinity binding to RARα, RARβ and RARγ receptors such that RAR-mediated signalling is essentially inhibited and/or RAR mediated repression is enhanced leading to significantly increased chondrogenesis. Such compositions may comprise;
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ;
iii) a mixture of i) and ii).

The pan-antagonist compositions of the invention can be used either in vitro or in vivo and have similar effects on cells whether used in vitro or in vivo.

Any RAR pan-antagonist compound having a high binding affinity to RARα, RARβ and RARγ may be used in the present invention as is understood by one of skill in the art. It is generally understood that such compound stimulates chondrogenesis both in vivo and in vitro. As well in general, the present invention encompasses and any agent which demonstrates RAR pan-antagonist activity. Suitable pan-antagonist compounds for use in the present invention may be selected from AGN194310 and those disclosed in U.S. Pat. No. 6,313,168 (the contents of which is incorporated herein by reference in its entirety).

Any RAR antagonist compound having a high binding affinity to individual RARα, RARβ or RARγ may be used in the compositions of the present invention. As well in general, the present invention encompasses and any agent which demonstrates RAR antagonist activity. Mono- or di-fluoro substituted methylchromenes are useful compounds such as AGN 194301, (2-Fluoro-4-[(1-(8-bromo-2,2-dimethyl-4-(4-methylphenyl)-2-H-chromen-6-yl)-methanoyl)-amino]-benzoic acid) which is a potent antagonist of RARα with a lower affinity for RARβ and RARγ.

The RAR pan-antagonist compounds and RAR compounds of the invention may be synthesized by conventional chemical synthetic methods. For example, AGN 194301 may be synthesised as described in Teng et al., (1997), J. Med. Chem., 40, 2445-2451. Those of ordinary skill in the art are able to screen candidate compounds to identify compounds having such an RAR antagonist or pan-antagonist profile by methods available in the scientific literature, for example as described in Teng et al., (supra). Means for determining antagonist or pan-antagonist activity of a given agent or compounds is also disclosed for example in WO 93/11755 (the contents of which are incorporated herein by reference).

One skilled in the art would readily understand that several different types of RAR pan-antagonists and antagonists specifically for RARα, RARβ and RARγ other than those described specifically herein are suitable for use in the present invention. Suitable RAR antagonists are taught for example in WO 9933821, WO 9924415, U.S. Pat. Nos. 5,877,207, 5,514,825, 5,648,514, 5,728,846, 5,739,338, 5,763,635, 5,808,124, 5,773,594, 5,760,276, 5,776,699, and JP 10114757 (the disclosures of which are incorporated herein by reference). Such antagonist agents include but are not limited to AGN 193109, AGN 190121, AGN 194574, AGN 193174, AGN 193639, AGN 193676, AGN 193644, SRI 11335, Ro 41-5253, Ro 40-6055, CD 2366, BMS 185411, BMS 189453, CD-2665, CD 2019, CD 2781, CD 2665, CD 271. Other suitable RAR antagonists for use in the present invention include those disclosed in Kaneko et al., 1991; Apfel et al., 1992; Eyrolles et al., 1994; Yoshimura et al., 1995; Eckharat and Schmitt, 1994; and Teng et al., 1997 (the disclosures of which are incorporated herein by reference).

The chondrogenesis-stimulating RAR pan-antagonists compositions of the invention are useful for the treatment and management of cartilage problems or abnormalities resulting from disease or trauma in vertebrates, including humans and other mammals. They may be used in several therapeutic applications where increased chondrogenesis is desired, for stimulation of associated skeletal development and for slowing of cartilage disease progression. Therapeutic applications of these pan-antagonist compositions include the stimulation of new cartilage formation and accelerate associated bone repair.

A RAR pan-antagonist pharmaceutical composition as herein defined may be applied locally to a fracture site, for example by means of a biodegradable sponge, gel, coating or paste. A suitable gel for use would be a collagen type gel such as collagen I.

The RAR pan-antagonist compositions of the present invention may also be used for the treatment of orthopedic or dental implants to enhance or accelerate osseous integration. The RAR pan-antagonist composition may be directly applied locally to the site of desired osseous integration or alternatively as a coating on implants.

The RAR pan-antagonist compositions of the invention may also be used for promoting in vivo integration of implantable prosthetic devices. In general, the RAR pan-antagonist compositions of the invention may be applied to synthetic bone grafts for implantation whereby the pan-antagonist composition stimulates cartilage formation and indirectly bone formation. The compositions thus have numerous applications in the orthopedic industry. In particular, there are applications in the fields of trauma repair, spinal fusion, reconstructive surgery, maxillo-facial surgery and dental surgery. The ability of the RAR pan-antagonist compositions to stimulate local natural bone growth provides stability and rapid integration, while the body's normal cell-based bone remodeling process slowly resorbs and replaces a selected implant with natural bone. Implants suitable for in vivo use are generally known to those skilled in the art.

The RAR pan-antagonist compositions of the invention may be used for cartilage and skeletal reconstruction. In such an application, the compositions can be used for ex vivo tissue engineering of cartilage or skeletal tissue for implantation in a vertebrate. Cells can be treated with a RAR pan-antagonist composition of the invention during osteochondral autograft or allograft transplantations (Minas et al., (1997), Orthopedics, 20, 525-538). In autograft transplantations, chondrogenic cells or cells with chondrogenic potential (i.e. precursor cells of the chondrocyte lineage) are removed from a patient (e.g. from a rib) and used to fill a cartilaginous lesion. An alternative method involves expanding these cells in vitro, then implanting them into a cartilaginous lesion. A pharmaceutical pan-antagonist composition of the invention would be used to treat the cells in in vitro culture prior to engraftment and/or after engraftment through intra-articular injection. The use of the RAR pan-antagonist compositions of the invention may eliminate the pain and costs associated with the bone harvest procedure required in autograft transplants. Furthermore, the RAR pan-antagonist compositions can be made synthetically thus reducing the possibility of transmission of infection and disease, as well as diminishing the likelihood of immunological rejection by the patient.

More specifically, for in vitro and ex vivo tissue engineering use, one skilled in the art may apply a RAR pan-antagonist composition of the invention to a desired culture of cells. Representative cell cultures are those precursor cells of chondrocyte lineage that are capable of differentiating into chondroblasts, such as but not limited to embryonic stem cells, adult stem cells and chondroprogenitor cells derived from bone, bone marrow or blood. Such cells may also include dedifferentiated cells. Cell cultures may be maintained until a desired physiological result is achieved after which the cells are administered by various conventional methods to patient at a desired tissue site. Alternatively, such cultured treated cells may be applied or growth within to an implant or within an implant or prosthetic device and further cultured in vitro to allow for chondrogenesis to take place prior to patient implantation.

The RAR pan-antagonist compositions of the present invention may also be used for the treatment of arthritis such as rheumatoid arthritis. To reverse or slow degenerative joint disease characterized by cartilage degeneration, a RAR pan-antagonist composition may be applied locally through intra-articular injection or in combination with a viscosupplement. The composition may be provided in either a fast-release or slow-release formulation. Such compositions have use in patients with degenerative hip or knee joints, for example.

In general, the RAR pan-antagonist compositions of the invention may be used to stimulate in vitro chondrogenesis from mesenchymal precursor cells (i.e. any precursor cells of chondrocyte lineage) and in vitro formation of chondrocytes. Such cell culture materials and methods are known to those skilled in the art and are also described herein in the examples. Cells and tissues treated with a selected RAR pan-antagonist composition in vitro can be used therapeutically in vivo or alternatively for in vitro cellular assay systems.

The pharmaceutical RAR pan-antagonist compositions of the invention may be used in combination with other chondrogenic stimulators, e.g. bone morphogenetic proteins (BMPs) especially BMP-2 and BMP4, osteogenic proteins (OPs) such as OP-1 and/or cytokines to enhance and/or maintain the effects of the compositions. Both BMPs and OPs are proteins belonging to the TGF-β superfamily which represent proteins involved in growth and differentiation as well as tissue morphogenesis and repair. It is also understood that the RAR pan-antagonist compositions of the invention may additionally comprise other chondroinductive agents or factors, defined as any natural or synthetic organic or inorganic chemical or biochemical compound, or mixture of compounds which stimulate chondrogenesis. It is further understood that the RAR pan-antagonist compositions of the invention may also comprise other growth factors known to have a stimulatory effect on cartilage growth and formation. Examples of possible compounds for use with the compositions of the invention may also include for example, calcium preparations, calcitonin preparations, sex hormones (e.g. estrogen, estradiol), prostaglandin A1, bisphosphonic acids, ipriflavones, fluorine compounds (e.g. sodium fluoride), vitamin K, fibroblast growth factor (FGF), platelet-derived growth factor (PDGF), transforming growth factor (TGF-β), insulin-like growth factors 1 and 2 (IGF-1,2), parathyroid hormone (PTH), epidermal growth factor (EGF) and leukemia inhibitory factor (LIP).

Those of ordinary skill in the art are familiar with various methods of formulating pharmaceutical compositions for local administration in diseases such as arthritis. For example, Adams et al., (1995), Osteoarthritis & Cartilage, 3, 213-225, describes viscosupplementation in osteoarthritis; Wozney et al., (1998), Clin. Ortho. Rel. Res., 346, 26-37, describes delivery methods used for BMPs to effect bone repair and formation. These formulation methods may be employed to prepare the RAR pan-antagonist compositions of the invention.

For therapeutic applications in accordance with the present invention the RAR pan-antagonists are incorporated into pharmaceutical compositions formulated for oral or parenteral administration, the latter route including intravenous and subcutaneous administration. Parenteral administration may be by continuous infusion over a selected period of time. As such, the compositions may be provided as tablets, pills, capsules, solutions, suspensions, creams, gels, and the like.

An RAR pan-antagonist composition may be orally administered with an inert diluent or with an assimilable edible carrier, or it may be enclosed in hard or soft shell gelatin capsules, compressed into tablets or incorporated directly with the food of the diet. For oral therapeutic administration, the RAR pan-antagonist composition may be incorporated with excipient and used in the form in ingestible tablets, buccal tablets, troches, capsules, elixirs, suspensions, syrups, wafers, and the like.

In one embodiment of the present invention is provided a pharmaceutical pan-antagonist composition for administration to subjects (i.e. any mammal or vertebrate) in a biologically compatible form suitable for administration in vivo for treating abnormal chrondrogenesis and associated skeletal development. The composition comprises a safe and effective amount of a selected RAR pan-antagonist alone or combination of selected RAR antagonist, further in combination with other agents and/or pharmaceutically acceptable carriers. The composition may be administered to any living organism including humans and animals. By “safe and effective amount” as used herein is meant providing sufficient potency in order to decrease, prevent, ameliorate or treat a chondrogenesis or skeletal disorder affecting a subject while avoiding serious side effects. A safe and effective amount will vary depending on the age of the subject, the physical condition of the subject being treated, the severity of the disorder, the duration of treatment and the nature of any concurrent therapy. Dosage regima may be adjusted to provide the optimum therapeutic response. For example, several divided doses may be administered daily or the dose may be proportionally reduced as indicated by the exigencies of the therapeutic situation. The compositions are preferably in the form of a unit dose and will usually be administered as a dose regimen that depends on the particular tissue treatment.

The compositions described herein can be prepared by known methods for the preparation of pharmaceutically acceptable compositions which can be administered to subjects, such that an effective quantity of the active substance is combined in a mixture with a pharmaceutically acceptable carrier. By pharmaceutically acceptable carrier as used herein is meant one or more compatible conventional solid or liquid delivery systems as are well known in the art. Some examples of pharmaceutically acceptable carriers are sugars, starches, cellulose and its derivatives, powdered tragacanth, malt, gelatin, collagen, talc, stearic acids, magnesium stearate, calcium sulfate, vegetable oils, polyols, agar, alginic acids, pyrogen-free water, isotonic saline, phosphate buffer, and other suitable non-toxic substances and medicinal agents used in pharmaceutical formulations. Other excipients such as wetting agents and lubricants, tableting agents, stabilizers, anti-oxidants and preservatives are also contemplated. Suitable carriers are further described for example in Remington's Pharmaceutical Sciences (Remington's Pharmaceutical Sciences, Mack Publishing Company, Easton, Pa., USA 1985). On this basis the compositions include, albeit not exclusively, solutions of the substance in association with one or more pharmaceutically acceptable vehicles or diluents, and contained in buffered solutions with a suitable pH and iso-osmotic with the physiological fluids.

The RAR pan-antagonist compositions of the invention can be provided as a liquid for local administration at a desired tissue site such as by injection. Alternatively, the compositions of the invention can be provided encapsulated for administration to a desired tissue site. The RAR pan-antagonist composition may may be provided as a solution or emulsion contained within phospholipid vesicles called liposomes. The liposomes may be unilamellar or multilamellar and are formed of constituents selected from phosphatidylcholine, dipalmitoylphosphatidylcholine, cholesterol, phosphatidylethanolamine, phosphatidylserine, demyristoylphosphatidylcholine and combinations thereof. The multilamellar liposomes comprise multilamellar vesicles of similar composition to unilamellar vesicles, but are prepared so as to result in a plurality of compartments in which the silver component in solution or emulsion is entrapped. Additionally, other adjuvants and modifiers may be included in the liposomal formulation such as polyethyleneglycol, or other materials.

It is understood by those skilled in the art that any number of liposome bilayer compositions can be used in the composition of the present invention. Liposomes may be prepared by a variety of known methods such as those disclosed in U.S. Pat. No. 4,235,871 and in RRC, Liposomes: A Practical Approach. IRL Press, Oxford, 1990, pages 33-101.

The liposomes containing a RAR pan-antagonist composition may have modifications such as having non-polymer molecules bound to the exterior of the liposome such as haptens, enzymes, antibodies or antibody fragments, cytokines and hormones and other small proteins, polypeptides or non-protein molecules which confer a desired enzymatic or surface recognition feature to the liposome. Surface molecules which preferentially target the liposome to specific organs or cell types include for example antibodies which target the liposomes to cells bearing specific antigens. Techniques for coupling such molecules are well known to those skilled in the art (see for example U.S. Pat. No. 4,762,915 the disclosure of which is incorporated herein by reference). Alternatively, or in conjunction, one skilled in the art would understand that any number of lipids bearing a positive or negative net charge may be used to alter the surface charge or surface charge density of the liposome membrane.

The liposomes can also incorporate thermal sensitive or pH sensitive lipids as a component of the lipid bilayer to provide controlled degradation of the lipid vesicle membrane.

For systemic application by intravenous delivery, it may be beneficial to encapsulate the RAR pan-antagonist composition within sterically-stabilized liposomes which exhibit prolonged circulation time in blood. The sterically stabilized liposomes are produced containing polyethylene glycol as an essential component of their surface and the method of making such liposomes is known to those skilled in the art. The size of the liposomes can be selected based on the intended target and route of administration. Liposomes of between about 10 nm to 300 nm may be suitable. Furthermore, the composition of the present invention may include liposomes of different sizes.

While the composition of the present invention may be encapsulated for administration by liposomes, it is understood by those skilled in the art that other types of encapsulants may also be used to encapsulate the compositions of the invention. Microspheres including but not limited to those composed of ion-exchange resins, crystalline ceramics, biocompatible glass, latex and dispersed particles are suitable for use in the present invention. Similarly, nanospheres and other lipid, polymer or protein materials can also be used.

The RAR pan-antagonist compositions of the present invention may be dispersed in an implantable biocompatible carrier that functions as a suitable delivery or support system. Suitable examples of biocompatible sustained release carriers include semi-permeable polymer matrices in the form of shaped implantable articles such as polylactides, copolymers of L-glutamic acid, ethyl-L-glutamate, poly(2-hydroyethyl-methacrylate) or ethylene vinyl acetate. Such matrices can be fabricated to have the RAR antagonist incorporated therein and be of a selected pore size to permit chondroprogenitor cells and skeletal progenitor cells to migrate within. The selected carrier material may also comprise a biodegradable, synthetic or synthetic-organic matrix such as hydroxyapatite, collagen, tricalcium phosphate or various copolymers of glycolid, lactic and butyric acid.

The RAR pan-antagonist composition of the present invention may also be used with demineralized allogenic bone and demineralized xenogenic bone optionally treated with fibril modifying agents. Furthermore, the composition may be provided with a mechanical or suitable physical device, influence or force such that it functions to promote chondrogenesis and skeletal development either in vitro or in vivo.

In summary, RAR pan-antagonists have important clinical therapeutic uses for treatment of cartilage and associated bone development defects. RAR pan-antagonists are useful as compositions can be used to provide such treatment both in vitro and in vivo to treat a variety of conditions as a result of trauma, genetic disease or degenerative disease negatively affecting cartilage and associated bone development and maintenance.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Expression Plasmids and Reporter Constructs

The Sox9 responsive reporter (herein referred to as the Sox9 reporter) was generated by subcloning a fragment containing a reiterated (4×48) Sox9 binding sequence coupled to the mouse Col2a1 minimal promoter (−89 to +6) into pGL3. The fragment containing the 4×48 repeat and minimal promoter was isolated as a BamH I/Hind III fragment from the original 4×48-p89Luc reporter plasmid previously described (Lefebvre et al., 1997), and was subcloned into the BgI II and Hind III sites of pGL3-basic (Promega) to generate pGL3(4×48). The reporter pW1-Col2-Luc was generated from the original p309i(182×2)βgeoCol2a1 (Zhou et al., 1995) by subcloning the regulatory region (consisting of a 309-bp promoter region and two tandem repeats of a 182-bp intron-1 fragment) into pW1 (Balkan et al., 1992) as an EcoR I/BamH I fragment. A BgI II fragment containing the luciferase gene isolated from pJD205 (de Wet et al., 1987) was subcloned into the BamH I site of pW1-Col2 to generate pW1-Col2-Luc. The pcDNA3-hSox9 expression vector was as described (Lefebvre et al., 1997). To generate a mutated form of hSox9, hSox9 was subcloned into pKSII (Stratagene) and serine 181 was replaced with alanine, using the Quick-Change XL system (Stratagene) with the following overlapping primers: 5′-GCCGCGGCGGAGGAAGGCGGTGAAGAACGGGCAGG-3′, and 5′-CCTGCCCGTTCTTCACCGCCTTCCTCCGCCGCGGC-3′. Following mutagenesis, the Sox9 mutant and wt Sox9 were subcloned into pcDNA3, and the serine-alanine conversion was confirmed by sequencing.

The dominant-negative versions of RARα and RXRα were generated as EGFP fusions containing C-terminal truncations at amino acid positions 403 and 449, respectively (Damm et al., 1993; Feng et al., 1997). A BgI II restriction endonuclease site was incorporated into the primers to facilitate cloning and to allow for an in-frame fusion to pEGFP-N1 (Clontech). Internal primers used for truncation of the receptors were: 5′-AG ATC TGG GAT CTC CAT CTT CM TG-3′ for RARα and 5′-CAG ATC TCC GAT GAG CTT GM GM G-3′, for RXRα. For expression in cells, receptor-EGFP fusion constructs were cloned into the mammalian expression plasmid pSG5 (Stratagene). EGFP-N1 was initially subcloned into the pSG5 vector followed by the corresponding truncated receptor to generate pSG5-dnRARαEGFP and pSG5-dnRXRαEGFP.

Constitutively active versions of RARα and RXRα were subcloned into pSG5HS as Hind III/Spe I fragments isolated from the constructs described (Underhill et al., 1994). These receptors contain carboxy terminal fusions to the acidic activation domain of VP16 (Underhill et al., 1994). The constitutively active version of MKK6 used here was the previously described pcDNA3-HA-MKK6E (Han et al., 1996). Expression plasmids, pcDNA3-p38β-Flag, and pcDNA3-p38β2-Flag, were used to express p38α and p38β2 in mesenchymal cells (Enslen et al., 1998). To activate the PKA pathway, pCMV-PKA (Clontech) which contains the catalytic subunit of PKA was used. To follow activation of ATF2 and CREB, constructs containing the transactivation domain of these transcription factors fused to the DNA binding domain of GAL4 (pFA-ATF2 and pFA-CREB) were used. (Stratagene). The pFA-ATF2 and pFA-CREB plasmids were co-transfected with pG5-Luc, a reporter containing five copies of a GAL4 DNA binding element upstream of a TATA box and the luciferase gene (Stratagene). PCMX-N-CoR and pCMX-GAL4/N-CoR (2174-2453) (referred to as pG/N-CoR(2174-2453) herein) consists of the DNA binding domain of GAL4 fused to the 3′ region of N-CoR encompassing amino acids 2174-2453, as described (Heinzel et al., 1997).

Reporter vectors from Systems 1 and 2 of Clontech's Mercury Pathway Profiling Systems were used to identify pathways downstream of retinoid signaling. These systems are sets of vectors that contain distinct cis-acting enhancer elements upstream of a TATA box and the luciferase gene.

### Example 2

### Establishment and Transient Transfection of Primary Limb Mesenchymal Cultures

Limb mesenchymal cells were harvested from embryonic age 11.25-11.75 mouse embryos as previously described (Weston et al., 2000). The cells were resuspended at a density of about 2.5×10⁷ cells/ml prior to transfections, otherwise they were resuspended at 1.5×10⁷ cells/ml. For transfection purposes, cells were mixed with a DNA/FuGene6 mixture in a 2:1 ratio. FuGene6-DNA mixtures were prepared according to the manufacturer's instructions (Roche Biomolecular). Briefly, 1 μg of reporter, 1 μg of expression vector and 0.05 μg of pRLSV40 (Promega) were mixed for a total of about 2 μg DNA in 100 μL of media and FuGene6. Fifty microlitres of the DNA mixture was transferred into a sterile 1.5 ml eppendorf tube, followed by 100 μl of cells. Cells were gently triturated and 10 μl was used to seed each single well of a 24-well culture dish. After 1.5 hrs in a humidified CO₂ incubator, 1 ml of media containing compounds of interest was added to each well and was subsequently replaced 24 hours following transfection. All-trans RA (at-RA; Sigma), AGN193836 (Teng et al., 1996), AGN194301 (Teng et al., 1997) and AGN194310 (Johnson et al., 1999)(Allergan Inc.) were dissolved in 95% ethanol. SB202190 and SB203580 (Calbiochem) and H89 (Sigma) were dissolved in dimethyl sulfoxide (DMSO) (BDH). 8-(4-chlorophenylthio)-cAMP (pCPT-cAMP, Sigma) was dissolved in water just prior to use.

Analysis of reporter gene activity using the Dual Luciferase Assay System was done following the manufacturer's instructions (Promega). Briefly, approximately 48 hours following the transfections, cells were washed once with PBS and lysed in 100 μl of Passive Lysis Buffer for 20 minutes. Firefly and renilla luciferase activities were determined using 40 μl of lysate in a 96-well plate-reading luminometer (Molecular Devices). Alcian blue staining of cultures was carried out as described previously (Cash et al., 1997).

### Example 3

### Cell Culture and Transient Transfection of Cell Lines

COS P7 cells were maintained in Dulbecco's Modified Eagle's Medium containing 10% fetal bovine serum (FBS) (Gibco-BRL) and antibiotics. C5.18 cells, subcloned from the parental chondroblast clone RCJ 3.1C5 (Grigoriadis et al., 1996), were maintained in α-MEM supplemented with 15% FBS and antibiotics. Articular chondrocytes were derived from the knee joints of 1-day-old Sprague-Dawley rats. Briefly, 1-2 mm cartilage fragments from the femoral condyles were isolated, washed 3 times in sterile phosphate-buffered saline (PBS), and digested with 0.3% Collagenase P (Worthington Biochemical Corporation) at 37° C. for 4 hours, adding fresh Collagenase P after the first 30 minutes. Following digestion, cells were filtered through a cell strainer (70 μm, Falcon) to obtain a single cell suspension. PBS/Collagenase was removed, cells were resuspended at approximately 1.5×10⁵ cells/ml, and 6 ml were transferred to a T-25 tissue culture flask. Upon reaching confluence, cells were transferred to a T-75 flask.

For transient transfections, the cells described were plated at 5×10⁴ cells/well in 12-well tissue culture plates approximately 24 hours prior to transfection. FuGene6 transfection reagent was used according to manufacturer's instructions (Roche Biomolecular). Each well of cells was transfected with a FuGene-DNA mixture containing a total of 0.5 μg DNA, comprised of 0.3 μg of reporter, 0.2 μg of expression vector, and 0.05 μg of pRLSV40. Media was changed approximately 24 hours following transfection, and luciferase assays were carried out approximately 48 hours following transfection. Luciferase assays were done as described above with the exception of using 200 μl/well of Passive Lysis Buffer (Promega) to obtain cell extracts.

### Example 4

### Northern Blot Analysis and Real-Time Quantitative PCR

Northern blots were carried out using total RNA from limb mesenchymal cultures, as previously described (Weston et al., 2000). Briefly, total RNA was extracted from cells cultured for 2, 4, 6 or 8 days. Cells were treated with media alone or with AGN 194301. Synthesis of the Col2a1 cDNA fragment used was as described previously (Weston et al., 2000). The Sox9 cDNA probe was made using an EST clone, GenBank accession number Al594348 (Research Genetics). The Sox9 fragment was released from pT7T3 using Eco RI and Not I.

To monitor changes in transcript levels of Sox9 and Col2a1, quantitative real-time PCR was carried out using the 7900HT Sequence Detection System (Applied Biosystems). Primers and TaqMan-minor groove binding (MGB)-probes were designed using PrimerDesigner 2.0 (Applied Biosystems).

The following primer/probe sets were used for detection of Col2a1: forward primer, 5′-GGCTCCCMCACCGCTMC, reverse primer, 5′-GATGTTCTGGGAGCCCTCAGT, probe 6FAM-5′-CAGATGACTTTCCTCCGTC-MGBNFQ. Sox9 transcripts were detected using the forward primer, 5′-CATCACCCGCTCGCAATAC, reverse primer, 5′-CCGGCTGCGTGACTGTAGTA, and probe, 6FAM-5′-ACCATCAGMCTCCGGCT-MGBNFQ.

Primer and probe concentrations were optimized according to the manufacturer's instructions. Total RNA was isolated from primary cultures as described above, and was treated with amplification-grade DNase I (Invitrogen). Quantification was carried out using 4 ng of total RNA and the expression of Sox9 or Col2a1 relative to endogenous rRNA was determined using TaqMan Ribosomal Control Reagents (Applied Biosystems) and the comparative C_{T} method as described in User Bulletin #2 (Applied Biosystems).

### Example 5

### Statistical Analysis

All luciferase assays were performed a minimum of three times using separate preparations of primary cells each time. Each transfection or treatment was carried out in quadruplicate for all experiments, with the exception of the COS cell transfections which were performed in triplicate. Real-time PCR analysis was carried out using RNA from two separate preparations, with treatments done in triplicate for each preparation. All luciferase reporter and expression data was analyzed by analysis of variance (ANOVA), followed by a Bonferroni post-test for multiple comparisons using GraphPad Prism, Version 2.0 (GraphPad Software Inc., San Diego, Calif.). One representative experiment is shown for all luciferase and expression results.

Although preferred embodiments of the invention have been described herein in detail, it will be understood by those skilled in the art that variations may be made thereto without departing from the spirit of the invention or the scope of the appended claims.

### REFERENCES

Balkan, W., M. Colbert, C. Bock, and E. Linney. 1992. Transgenic indicator mice for studying activated retinoic acid receptors during development. Proc Natl Acad Sci U S A. 89:3347-51.
Bell, D. M., K. K. Leung, S. C. Wheatley, L. J. Ng, S. Zhou, K. W. Ling, M. H. Sham, P. Koopman, P. P. Tam, and K. S. Cheah. 1997. SOX9 directly regulates the type-II collagen gene. Nat Genet. 16:174-8.
Bi, W., J. M. Deng, Z. Zhang, R. R. Behringer, and B. de Crombrugghe. 1999. Sox9 is required for cartilage formation. Nat Genet. 22:85-9.
Cash, D. E., C. B. Bock, K. Schughart, E. Linney, and T. M. Underhill. 1997. Retinoic acid receptor alpha function in vertebrate limb skeletogenesis: a modulator of chondrogenesis. J Cell Biol. 136:445-57.
Cohlan, S. Q. 1953. Excessive intake of vitamin A as a cause of congenital anomalies in the rat. Science. 117:535-536.
Damm, K., R. A. Heyman, K. Umesono, and R. M. Evans. 1993. Functional inhibition of retinoic acid response by dominant negative retinoic acid receptor mutants. Proc Natl Acad Sci U S A. 90:2989-93.
de Wet, J. R., K. V. Wood, M. DeLuca, D. R. Helinski, and S. Subramani. 1987. Firefly luciferase gene: structure and expression in-mammalian cells. Mol Cell Biol. 7:725-37.
Dolle, P., E. Ruberte, P. Kastner, M. Petkovich, C. M. Stoner, L. J. Gudas, and P. Chambon. 1989. Differential expression of genes encoding alpha, beta and gamma retinoic acid receptors and CRABP in the developing limbs of the mouse. Nature. 342:702-5.
Enslen, H., J. Raingeaud, and R. J. Davis. 1998. Selective activation of p38 mitogen-activated protein (MAP) kinase isoforms by the MAP kinase kinases MKK3 and MKK6. J Biol Chem. 273:1741-8.
Feng, X., Z. H. Peng, W. Di, X. Y. Li, C. Rochefte-Egly, P. Chambon, J. J. Voorhees, and J. H. Xiao. 1997. Suprabasal expression of a dominant-negative RXR alpha mutant in transgenic mouse epidermis impairs regulation of gene transcription and basal keratinocyte proliferation by RAR-selective retinoids. Genes Dev. 11:59-71.
Ferrara, F. F., F. Fazi, A. Bianchini, F. Padula, V. Gelmetti, S. Minucci, M. Mancini, P. G. Pelicci, F. Lo Coco, and C. Nervi. 2001. Histone deacetylase-targeted treatment restores retinoic acid signaling and differentiation in acute myeloid leukemia. Cancer Res. 61:2-7.
Finnin, M. S., J. R. Donigian, A. Cohen, V. M. Richon, R. A. Rifkind, P. A. Marks, R. Breslow, and N. P. Pavletich. 1999. Structures of a histone deacetylase homologue bound to the TSA and SAHA inhibitors. Nature. 401:188-93.
Foster, J. W., M. A. Dominguez-Steglich, S. Guioli, G. Kowk, P. A. Weller, M. Stevanovic, J. Weissenbach, S. Mansour, I. D. Young, P. N. Goodfellow, and et al. 1994. Campomelic dysplasia and autosomal sex reversal caused by mutations in an SRY-related gene. Nature. 372:525-30.
Grigoriadis, A. E., J. N. Heersche, and J. E. Aubin. 1996. Analysis of chondroprogenitor frequency and cartilage differentiation in a novel family of clonal chondrogenic rat cell lines. Differentiation. 60:299-307.
Hale, F. 1935. The relation of vitamin A to anophthalmos in pigs. Am J Othamol. 18:1087-1093.
Han, J., J. D. Lee, Y. Jiang, Z. Li, L. Feng, and R. J. Ulevitch. 1996. Characterization of the structure and function of a novel MAP kinase kinase (MKK6). J Biol Chem. 271:2886-91.
Healy, C., D. Uwanogho, and P. T. Sharpe. 1999. Regulation and role of Sox9 in cartilage formation. Dev Dyn. 215:69-78.
Huang, W., X. Zhou, V. Lefebvre, and B. de Crombrugghe. 2000. Phosphorylation of SOX9 by cyclic AMP-dependent protein kinase A enhances SOX9's ability to transactivate a Col2a1 chondrocyte-specific enhancer. Mol Cell Biol. 20:4149-58.
Johnson, A. T., L. Wang, A. M. Standeven, M. Escobar, and R. A. Chandraratna. 1999. Synthesis and biological activity of high-affinity retinoic acid receptor antagonists. Bioorg Med Chem. 7:1321-38.
Kalter, H., and J. Warkany. 1961. Experimental production of congenital malformations in strains of inbred mice by matemal treatment with hypervitaminosis. Am J Path. 38:1-21.
Kochhar, D. M. 1973. Limb development in mouse embryos. I. Analysis of teratogenic effects of retinoic acid. Teratology. 7:289-98.
Koide, T., M. Downes, R. A. Chandraratna, B. Blumberg, and K. Umesono. 2001. Active repression of RAR signaling is required for head formation. Genes Dev. 15:2111-21.
Kosher, R. A., S. W. Gay, J. R. Kamanitz, W. M. Kulyk, B. J. Rodgers, S. Sai, T. Tanaka, and M. L. Tanzer. 1986. Cartilage proteoglycan core protein gene expression during limb cartilage differentiation. Dev Biol. 118:112-7.
Koyama, Y., M. Adachi, M. Sekiya, M. Takekawa, and K. Imai. 2000. Histone deacetylase inhibitors suppress IL-2-mediated gene expression prior to induction of apoptosis. Blood. 96:1490-5.
Kwasigroch, T. E., and D. M. Kochhar. 1980. Production of congenital limb defects with retinoic acid: phenomenological evidence of progressive differentiation during limb morphogenesis. Anat Embryol (Berl). 161:105-13.
Lefebvre, V., W. Huang, V. R. Harley, P. N. Goodfellow, and B. de Crombrugghe. 1997. SOX9 is a potent activator of the chondrocyte-specific enhancer of the pro alpha1 (II) collagen gene. Mol Cell Biol. 17:2336-46.
Lefebvre, V., W. Huang, V. R. Harley, P. N. Goodfellow, and B. de Crombrugghe. 1997. SOX9 is a potent activator of the chondrocyte-specific enhancer of the pro alpha1 (II) collagen gene. Mol Cell Biol. 17:2336-46.
Leid, M., P. Kastner, and P. Chambon. 1992. Multiplicity generates diversity in the retinoic acid signalling pathways. Trends Biochem Sci. 17:427-33.
Mendelsohn, C., E. Ruberte, M. LeMeur, G. Morriss-Kay, and P. Chambon. 1991. Developmental analysis of the retinoic acid-inducible RAR-beta 2 promoter in transgenic animals. Development. 113:723-34.
Mollard, R., S. Viville, S. J. Ward, D. Decimo, P. Chambon, and P. Dolle. 2000. Tissue-specific expression of retinoic acid receptor isoform transcripts in the mouse embryo. Mech Dev. 94:223-32.
Nagy, L., H. Y. Kao, D. Chakravarti, R. J. Lin, C. A. Hassig, D. E. Ayer, S. L. Schreiber, and R. M. Evans. 1997. Nuclear receptor repression mediated by a complex containing SMRT, mSin3A, and histone deacetylase. Cell. 89:373-80.
Smits, P., P. Li, J. Mandel, Z. Zhang, J. M. Deng, R. R. Behringer, B. de Croumbrugghe, and V. Lefebvre. 2001. The transcription factors L-Sox5 and Sox6 are essential for cartilage formation. Dev Cell. 1:277-90.
Sugita, K., K. Koizumi, and H. Yoshida. 1992. Morphological reversion of sis-transformed NIH3T3 cells by trichostatin A. Cancer Res. 52:168-72.
Teng, M., T. T. Duong, A. T. Johnson, E. S. Klein, L. Wang, B. Khalifa, and R. A. Chandraratna. 1997. Identification of highly potent retinoic acid receptor alpha-selective antagonists. J Med Chem. 40:2445-51.
Teng, M., T. T. Duong, A. T. Johnson, E. S. Klein, L. Wang, B. Khalifa, and R. A. Chandraratna. 1997. Identification of highly potent retinoic acid receptor alpha-selective antagonists. J Med Chem. 40:2445-51.
Teng, M., T. T. Duong, E. S. Klein, M. E. Pino, and R. A. Chandraratna. 1996. Identification of a retinoic acid receptor alpha subtype specific agonist. J Med Chem. 39:3035-8.
Underhill, T. M., and A. D. Weston. 1998. Retinoids and their receptors in skeletal development. Mictosc Res Tech. 43:137-55.
Underhill, T. M., D. E. Cash, and E. Linney. 1994. Constitutively active retinoid receptors exhibit interfamily and intrafamily promoter specificity. Mol Endocrinol. 8:274-85.
Wagner, T., J. Wirth, J. Meyer, B. Zabel, M. Held, J. Zimmer, J. Pasantes, F. D. Bricarelli, J. Keutel, E. Hustert, and et al. 1994. Autosomal sex reversal and campomelic dysplasia are caused by mutations in and around the SRY-related gene SOX9. Cell. 79:1111-20.
Warkany, J., and S. Schraffenberger. 1946. Congenital malformations induced in rats by maternal vitamin A deficiency. I. Defects of the eye. Arch Ophthal. 35:150-169.
Weston, A. D., V. Rosen, R. A. S. Chandraratna, and T. M. Underhill. 2000. Regulation of skeletal progenitor differentiation by the BMP and retinoid signaling pathways. J Cell Biol. 148:679-90.
Wilson, J. G., C. B. Roth, and J. Warkany. 1953. An analysis of the syndrome of malformations induced by maternal vitamin A deficiency. Effects of restoration of vitamin A at various times during gestation. Am J Anat. 92:189-217.
Wong, C. W., and M. L. Privalsky. 1998. Transcriptional silencing is defined by isoform- and heterodimer-specific interactions between nuclear hormone receptors and corepressors. Mol Cell Biol. 18:5724-33.
Zhou, G., S. Garofalo, K. Mukhopadhyay, V. Lefebvre, C. N. Smith, H. Eberspaecher, and B. de Crombrugghe. 1995. A 182 bp fragment of the mouse pro alpha 1(II) collagen gene is sufficient to direct chondrocyte expression in transgenic mice. J Cell Sci. 108:3677-84.

1. An RAR pan-antagonist composition selected from the group consisting of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii),
wherein said composition further comprises a pharmaceutically acceptable carrier and wherein said composition is administered to a population of cells to essentially inhibit RAR mediated signalling and/or enhance RAR mediated repression leading to increased chondrogenesis.

2. The composition of claim 1, wherein said composition further comprises an agent selected from the group consisting of epidermal growth factor, fibroblast growth factor, platelet derived growth factor, transforming growth factor, parathyroid hormone, leukemia inhibitory factor, insulin-like growth factor, bone morphogenetic protein-2, bone morphogenetic protein-5, bone morphogenetic protein-5, osteogenin, sodium fluoride, estrogens, calcitonin, biphosphonates, calcium carbonate, prostaglandins, vitamin K and mixtures thereof.

3. The composition of claim 1, wherein said composition promotes chondrogenesis of precursor cells of chondrocyte lineage.

4. The composition of claim 3, wherein said composition is a solution, suspension, gel, matrix, film, paste, pill, tablet or encapsulated within liposomes.

5. The composition of claim 4, wherein said composition further comprises excipients, preservatives, solubilizers, buffering agents, albumin, lubricants, fillers, stabilizers and mixtures thereof.

6. The composition of claim 4, wherein said composition is administered locally or systemically.

7. The composition of claim 6, wherein said composition is administered via intra-articular injection.

8. The composition of claim 4, wherein said composition is used in conjunction with a device selected from the group consisting of implantable mechanical physical device, implantable biodegradable carrier; implantable biodegradable synthetic carrier, implantable prostheses, implantable demineralized allogenic bone and implantable demineralized xenogenic bone.

9. The use of a composition of claim 1, for the manufacture of a medicament for the stimulation of chondrogenesis.

10. A method for stimulating chondrogenesis in vivo or in vitro, said method comprising contacting a precursor cell of chondrocyte lineage with a composition of claim 1.

11. (Currently Amended) The use of a RAR pan-antagonist composition selected from the group consisting of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii),
wherein said composition further comprises a pharmaceutically acceptable carrier and wherein said composition is administered to a population of cells to essentially inhibit RAR mediated signalling and/or enhance RAR mediated repression leading to increased chondrogenesis, said use being for a method selected from the group consisting of:
a) promoting in vivo integration of an implantable prosthetic device, into a target cartilage tissue of a vertebrate by providing said RAR pan-antagonist composition on a surface of the prosthetic device and implanting the device in a vertebrate at a site where the target cartilage tissue and surface of the prosthetic device are maintained at least partially in contact for a time sufficient to permit enhanced tissue growth between the target cartilage tissue and the device; and
b) aiding the attachment of implantable prosthesis at cartilageous sites and for maintaining the long term stability of the prostheses in vertebrates by coating selected regions of an implantable prosthesis with said RAR pan-antagonist composition and implanting the coated prosthesis into a cartilageous site wherein such implantation promotes the formation of new cartilage tissue.

12. A method of treating, ameliorating or repairing (a) a cartilage associated degenerative condition, (b) a skeletal defect and/or large segmental skeletal gap and (c) non-union fractures arising from trauma or surgery in a subject, said method comprising the step of administering a pharmaceutical composition as claimed in claim 1 to said subject.

13. The method of claim 12, wherein said condition is arthritis.

14. The method of claim 12, wherein said condition is degenerative joint disease.

15. The method of claim 12, wherein said RAR pan-antagonist composition is provided at the site of skeletal surgery wherein such delivery promotes the formation of new bone tissue.

16. The method of claim 12, wherein said RAR pan-antagonist composition is delivered at the site of the segmental skeletal gap or non-union fracture wherein such delivery promotes chondrogenesis which mediates the formation of new bone tissue.

17. A method of producing cartilage at a cartilage defect in vivo, said method comprising:
implanting into the defect a population of precursor cells of chondrocyte lineage which have been cultured in the presence of the composition of claim 1.

18. The method of claim 17, wherein said implanting is by intra-articular injection.

19. An implantable prosthetic device for repairing an orthopedic defect, injury or anomaly in a vertebrate, said device comprising:
(a) a prosthetic implant having a surface region implantable adjacent or within a target tissue; and
(b) RAR pan-antagonist composition incorporated on and/or within said prosthetic implant, said composition selected from the group consisting of:
i) a mixture of one or more RAR pan-antagonist compounds each of which has a high binding affinity to RARα, RARβ and RARγ;
ii) a mixture of at least two compounds that each have a high binding affinity to one of RARα, RARβ and RARγ; and
iii) a mixture of i) and ii),
wherein said composition further comprises a pharmaceutically acceptable carrier and wherein said composition essentially inhibits RAR mediated signalling and/or enhances RAR mediated repression leading to increased chondrogenesis.

20. A method for the ex vivo engineering of chondrocytes, said method comprising:
(a) culturing a population of precursor cells of chondrocyte lineage with a composition of claim 1 for a time sufficient to stimulate chondrogenesis; and
(b) implanting the cells from (a) directly into a desired site in a subject or applying the cells from (a) to a device selected from the group consisting of implantable mechanical physical device, implantable biodegradable carrier; implantable biodegradable synthetic carrier, implantable prostheses, implantable demineralized allogenic bone and implantable demineralized xenogenic bone, prior to implantation into a subject.
